SEQUENCE LISTING (1 sequence)

SEQ ID NO: 1 - protein, 17 aa, Homo sapiens
SSKITHRIHWESASLLR

(19) United States
(12) Patent Application Publication
(10) Pub. No.: US 2015/0285805 A1
(43) Pub. Date: Oct. 8, 2015

(54) BIOMARKERS OF MULTIPLE MYELOMA DEVELOPMENT AND PROGRESSION

(71) Applicants: OSPEDALE SAN RAFFAELE S.R.L., Milano (IT); FONDAZIONE CENTRO SAN RAFFAELE S.R.L., Milano (IT)

(72) Inventors: Simone Cenci, Milano (MI) (IT); Francesca Fontana, Milano (MI) (IT); Josè Manuel Garcia Manteiga, Milano (MI) (IT)

(21) Appl. No.: 14/440,782

(22) PCT Filed: Nov. 5, 2013
(86) PCT No.: PCT/EP2013/073067

§ 371 Date: May 5, 2015

Related U.S. Application Data
(60) Provisional application No. 61/722,555, filed on Nov. 5, 2012.

(51) Int. Cl. G01N 33/574

(52) U.S. Cl. CPC G01N 33/57426; G01N 2333/47; G01N 2333/46

(57) ABSTRACT

The present invention relates to a method for the prognosis of Symptomatic Multiple Myeloma (MM) and/or to monitor the response and/or the efficacy of a MM therapy comprising detecting and/or quantifying at least one marker, to relative kit and uses thereof and to relative microarray and use thereof. Markers include C3f peptide or a fragment thereof and/or other metabolites.

### TECHNICAL FIELD

The present invention relates to a method for the prognosis of Symptomatic Multiple Myeloma (MM) and/or to monitor the response and/or the efficacy of a MM therapy comprising detecting and/or quantifying at least one marker, to relative kit and uses thereof and to relative microarray and use thereof.

### BACKGROUND ART

Multiple Myeloma (MM) is a neoplastic disorder of plasma cells (PC), which typically grow at multiple foci in the bone marrow (BM), secrete monoclonal immunoglobulins (Ig), and induce end-organ damage leading to hypercalcemia, renal failure, anemia and bone lesions[1]. MM commonly originates from monoclonal gammopathy of undetermined significance (MGUS), an asymptomatic expansion of a PC clone occurring in 3% adults over 50 years, with 1% yearly risk of progression to symptomatic myeloma [1, 2]. An intermediate condition, smoldering myeloma (SMM) is defined by the presence of over 10% PC in the BM or serum monoclonal Ig (paraprotein or M-component) exceeding 3 g/dl [3] in the absence of symptoms [2, 4]. In light of the recent development of more effective therapies, the possibility to treat SMM patients is currently under investigation[5]. The variability in the timing of progression, however, warrants accurate risk stratification to be at the basis of treatment indication[5]. Moreover, as most MGUS patients will never develop myeloma, a difficult equilibrium needs to be achieved between the sustainability of follow-up and the capability to identify progression to active disease as early and efficiently as possible[2, 5].

The complete set of small metabolites within a biological system, or metabolome, results from the complex interaction between molecules, cells, and tissues. Unbiased and intrinsically integrative, metabolomics, the new “omics” of the post-genomic era, analyzes and quantifies at once all small metabolites with high potency and accuracy. Recently, metabolomics emerged as a powerful strategy to identify biomarkers of disease, and advance the understanding of molecular mechanisms of many disorders [6, 7].

Myeloma is believed to develop and progress by establishing vicious interactions with the BM multi-cellular milieu [8]. In keeping with a crucial role of the microenvironment, MGUS and SMM cells share many genetic abnormalities with MM cells[9], and exhibit extremely variable risk to become symptomatic[10]. Understanding the microenvironmental changes associated with myeloma would help identify biomarkers of prognostic value, and unveil disease mechanisms and potential therapeutic targets for future validation.

In search for markers of MM development and progression, the authors set out to exploit metabolic profiling to achieve an unbiased, comprehensive assessment of the extracellular milieu. The authors thus utilized BM aspirates from myeloma patients and individuals with MGUS to obtain the biofluid in closest proximity to the tumor, hereafter referred to as BM plasma. By excluding cells, this approach limits the effects of heterogeneity in sampling (e.g., from inefficient detachment of certain cell types) and avoids cell type selection biases. Moreover, by focusing on diffusible molecules, the authors also analyzed the metabolic profile of peripheral blood, less invasively collected, extending the study to age-matched healthy volunteers and larger patient numbers.

The authors analyzed 167 samples by an established UHPLCGC-MS (ultra-high performance liquid and gas chromatography followed by mass spectrometry) platform [7, 11, 12], leading to the identification of >300 metabolites. Dimensionality reduction methods [13] were employed to interrogate the dataset obtained, generating feature transformation-based scores and selecting candidate biomarkers. The ability of these metabolic scores to discriminate active MM from controls and correlate with BM PC counts was tested. Different feature selection analyses converged to consistent myeloma-associated metabolites as potential novel biomarkers. Interestingly, some have proposed functions in cancer growth and immune escape, but had not been previously reported to play a role in MM. An entire class of lipids decreased consistently in myeloma, and proved trophic to MM cells in vitro.

In all, the authors' work demonstrates that central and peripheral metabolomics is suitable for robustly defining the metabolic changes associated with development and progression of MM, leading to the identification of MM prognosis markers and pathways. Owing to its integrative nature, metabolomics proves an appropriate, powerful approach to study MM in its systemic complexity.

Patients may be classified into one of three myeloma categories:
Monoclonal Gammopathy of Undetermined Significance (MGUS)
Asymptomatic Multiple Myeloma or Smoldering Multiple Myeloma (SMM) or Indolent Multiple Myeloma (IMM)
Symptomatic Multiple Myeloma (MM)

The application WO2007038758 discloses a method for the diagnosis or prognosis of a systemic inflammatory condition in a patient comprising the step of measuring over time a plurality of amounts of total lysophosphatidylcholine in fluid or tissue of the patient to assess risk for the systemic inflammatory condition.

WO2007109881 describes a Lysophosphatidylcholine-related compounds, metabolites and N,N-dimethyl-lysophosphoethanolamine-related compounds have been claimed to be markers for diagnosing prostate cancer.

EP1866650 describes the value of the concentration of kynurenine in a body fluid as predictive marker for the detection of depression.

WO2011130385 describes the analysis of level of a marker such as kynurenine, or plurality of markers for determining if a subject has hepatocellular cancer (HCC).

The diagnosis of multiple myeloma (MM) relies on the presence of monoclonal Ig (M-protein) in the serum and/or urine, high bone marrow plasma cell counts, and end-organ damage (e.g., hypercalcemia, renal failure, anemia, bone disease). Clinical assessment relies on accurate physical evaluation, patient history, bone marrow aspiration, skeletal evaluation (total body X-ray or MRI) and various lab tests (including Complete Blood Count, comprehensive metabolic panel, urine, C-reactive protein and serum viscosity tests). Retrospective studies have shown that over 99% of MM evolve from MGUS, an asymptomatic frequent condition (−3% of the population over 50 years of age) associated with a 1% yearly risk of progression to MM. High M-protein levels (>3 g/dL) or bone marrow plasma cell counts (>10%) in absence of end-organ damage define SMM, a higher-risk precursor condition (70% progression within 10 years). Due to the severity of disease-defining symptoms and the availability of novel more effective treatments, early therapeutic intervention is currently under evaluation. Being progression poorly predictable, the need for accurate risk stratification and efficient monitoring is widely acknowledged. However, minimally invasive and accurate predictive markers are currently unavailable.

### SUMMARY OF THE INVENTION

The development of multiple myeloma relies on vicious interactions with the bone microenvironment, a deeper knowledge of which is needed to identify prognostic markers and potential therapeutic targets. To achieve an unbiased, comprehensive assessment of the extracellular milieu of myeloma, the authors performed metabolic profiling of patient-derived peripheral and bone marrow plasma by UHPLCGC-MS. In multivariate analyses, metabolic profiling of both peripheral and bone marrow plasma successfully discriminated active disease from control conditions (health, MGUS or remission), and correlated with bone marrow plasma cell counts. Independent disease vs. control comparisons consistently identified a number of metabolic alterations hallmarking active disease, including increased levels of the complement C3f peptide having the sequence SSKITHRIHWESASLLR (SEQ ID NO. 1), in particular of the fragment thereof comprising the sequence HWESAS (aa. 9 to 14 of SEQ ID NO. 1), in particular consisting of the sequence HWESASLL (aa. 9 to 16 of SEQ ID NO. 1), of specific aminoacid metabolites, and decreased lysophosphocholines. In the present invention the authors identify biomarkers of multiple myeloma development and progression both in peripheral and bone marrow plasma:
high level of the complement C3f peptide (or a fragment thereof comprising the sequence HWESAS) as marker i) of progression to myeloma from precursor conditions, MGUS and smoldering myeloma (SMM), and/or ii) of relapse in myeloma patients after treatment, and/or iii) of refractory myeloma, i.e., a myeloma that does not respond to therapeutic intervention;
low levels of lysophosphocho lines (LPC or glycerophosphocholines) as marker i) of progression to myeloma from precursor conditions, MGUS and smoldering myeloma (SMM), and/or ii) of relapse in myeloma patients after treatment and/or iii) of refractory myeloma, i.e., a myeloma that does not respond to therapeutic intervention;
high levels of pro-hydroxy-proline as marker of i) progression to myeloma from precursor conditions (MGUS, SMM), and/or of ii) relapse in myeloma patients after treatment and/or iii) of refractory myeloma, i.e a myeloma that does not respond to therapeutic intervention;
high levels of 3-hydroxykynurenine as marker of i) myeloma progression to myeloma from precursor conditions (MGUS, SMM), of ii) relapse in myeloma patients after treatment and/or iii) of refractory myeloma, i.e., a myeloma that does not respond to therapeutic intervention;
high level of sarcosine as a marker of myeloma or of high risk of progression from myeloma precursor condition (MGUS, SMM);
patients in complete remission or with very good partial response to treatment had significantly lower C3f and higher lysophosphocholine levels than active disease, i.e., newly diagnosed (pre-treatment), or relapsing and progressing despite treatment.

In particular, the inventors suggest that the combination of high levels of C3f, hydroxy-proline, 3-hydroxykynurenine, and sarcosine predict evolution to myeloma from precursor conditions (MGUS, SMM).

All markers can be determined for instance by mass spectrometry. The C3f peptide (sequence SSKITHRIHWESASLLR, SEQ ID NO. 1 and/or its fragments, including the form lacking the final arginine, also called des-Arginin-C3f or DRC3F, with sequence HWESASLL) can be detected by biochip-based methods, antibody-based techniques (such as ELISA), and mass spectrometry-based methods. Chromatography and UV-luminometric techniques can be used for the detection of pro-hydroxypro line, 3-hydroxykynurenine and sarcosine.

A dedicated assay may be developed for the targeted profiling of this very set of metabolites. The advantage of the invention resides in providing predictive markers of MM; individuals with monoclonal gammopathy of undetermined significance (MGUS) (3%>age 50) develop myeloma at a 1% yearly rate. 50% patients with asymptomatic (smoldering) myeloma develop symptomatic disease within 5 yrs. Patients that will evolve may benefit from early adoption of therapies, but predictive markers are currently unavailable. Metabolic biomarkers may predict imminent evolution to myeloma, and inform the design of dedicated clinical trials.

In vitro tests on cell lines and patient-derived myeloma cells revealed a previously unsuspected direct trophic function of lysophosphocho lines on malignant plasma cells. The authors' study proves metabolomics suitable both for studying the complex interactions of multiple myeloma with the bone marrow environment, and for identifying unanticipated disease markers to develop more accurate early diagnostic strategies.

It is therefore an object of the present invention a method for the prognosis of Symptomatic Multiple Myeloma (MM) and/or to monitor the response and/or the efficacy of a MM therapy comprising:
detecting and/or quantifying at least one marker selected from the group consisting of: 1-arachidonoylglycerophosphocholine, C3f peptide or a fragment thereof, 1-myristoylglycerophosphocholine, 2-palmitoylglycerophosphocholine, 1-pentadecanoylglycerophosphocholine 1-linoleoylglycerophosphocholine, 1-eicosatrienoylglycerophosphocholine, Creatinine, Glutaroyl carnitine, 2-linoleoylglycerophosphocholine, N-(2-furoyl)glycine, 1-palmitoleoylglycerophosphocholine, Citrate, Carnitine, Sarcosine (N-Methylglycine), 3-hydroxykynurenine, Xanthosine, 1-docosahexaenoylglycerophosphocholine, Testosterone sulfate, 1-palmitoylglycerophosphocho line, Glycerol 3-phosphate (G3P), Acetylcarnitine, N1-methyladenosine, Pro-hydroxy-pro, Urate, 7-alpha-hydroxy-3-oxo-4-cholestenoate (7-Hoca), Cysteine, 2-hydroxybutyrate (AHB), Cortisol, 1-oleoylglycerophosphoethanolamine, Butyrylcarnitine, Pyridoxate, Pseudouridine, 1-stearoylglycerophosphocholine, Palmitoyl sphingomyelin, 1-oleoylglycerophosphocholine, Creatine, 1-docosapentaenoylglycerophosphocholine, Gamma-glutamylphenylalanine, Catechol sulfate, 13-Hydroxyoctadecadienoate (13-HODE), 9-Hydroxyoctadecadienoate (9-HODE), Hexanoylcarnitine, 2-hydroxypalmitate, Indolepropionate, Oleoylcarnitine, Succinate, Tyrosine, Levulinate (4-oxovalerate), Adenosine 5′-monophosphate (AMP), Pyroglutamine, Pregn steroid monosulfate, Alpha-hydroxyisovalerate, Andro steroid monosulfate 2,3-methylhistidine, Lactate, Caprate (10:0), 3-hydroxyisobutyrate, Scyllo-inositol, N-acetyl-beta-alanine, 2-aminobutyrate, Phenyllactate (PLA), Heptanoate (7:0), Beta-hydroxyisovalerate, 3-methoxytyrosine, Deoxycarnitine, 1-palmitoylplasmenylethanolamine, 3-methyl-2-oxobutyrate, 3-phenylpropionate (hydrocinnamate), Propionylcarnitine, Pelargonate (9:0), Tryptophan, 2-stearoylglycerophosphocholine, Pregnenolone sulfate, Phosphate, N-acetylmethionine, Caprylate (8:0), N-formylmethionine, Cyclo(leu-pro), 1-heptadecanoylglycerophosphocholine, Pregnen-dioldisulfate, Acetylphosphate, Taurochenodeoxycholate, Arginine, Cholesterol, C-glycosyltryptophan, 4-androsten-3beta.17beta-diol disulfate 1, N-methyl proline, Stearoyl sphingomyelin, Mannose, 21-hydroxypregnenolone disulfate and 1-eicosadienoylglycerophosphocholine in a sample obtained from a subject;
optionally comparing the value of the quantified marker to a control value.

Preferably the C3f peptide or a fragment thereof is detected and/or quantified.

Preferably, the at least one marker is selected from the group consisting of: 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

Still preferably, the at least one marker is selected from the group consisting of: the C3f peptide or a fragment thereof, 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocho line, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

Preferably, the at least one marker is selected from the group consisting of: C3f peptide or a fragment thereof, 1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, Creatinine, Glutaroyl carnitine, 2-linoleoylglycerophosphocholine, N-(2-furoyl)glycine, 1-palmitoleoylglycerophosphocholine, Citrate, Carnitine, Sarcosine (N-Methylglycine), 3-hydroxykynurenine, Xanthosine, 1-docosahexaenoylglycerophosphocholine, Testosterone sulfate, 1-palmitoylglycerophosphocholine, Glycerol 3-phosphate (G3P), Acetylcarnitine, N1-methyladenosine, Pro-hydroxy-pro, Urate and 7-alpha-hydroxy-3-oxo-4-cholestenoate (7-Hoca). Still preferably, the at least one marker is selected from the group consisting of: C3f peptide or a fragment thereof, Sarcosine (N-Methylglycine), 3-hydroxykynurenine, Pro-hydroxy-pro, 1-arachidonoylglycerophosphocholine, 1-myristoylglycerophosphocholine, 2-palmitoylglycerophosphocho line, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocho line.

Still preferably, the at least one marker is selected from the group consisting of: C3f peptide or a fragment thereof, Sarcosine (N-Methylglycine), 3-hydroxykynurenine, Pro-hydroxy-pro, 1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocho line.

Yet preferably, the at least one marker is selected from the group consisting of: C3f peptide or a fragment thereof, Sarcosine (N-Methylglycine), 3-hydroxykynurenine and Pro-hydroxy-pro.

In a preferred embodiment the following markers are detected and/or measured: the C3f peptide Or a fragment thereof, 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocho line, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

In a preferred embodiment the following markers are detected and/or measured: C3f peptide or a fragment thereof, Sarcosine (N-Methylglycine), 3-hydroxykynurenine and Pro-hydroxy-pro.

In a still preferred embodiment the following further markers are detected and/or measured: 1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocho line.

Preferably the C3f peptide fragment comprises the sequence HWESAS. Still preferably, the C3f peptide fragment consists of sequence HWESASLL.

In a preferred embodiment the sample is blood, blood plasma or bone marrow plasma. Preferably the subject is affected by Monoclonal Gammopathy of Undetermined Significance (MGUS) or Asymptomatic Multiple Myeloma or Smoldering Multiple Myeloma (SMM) or Indolent Multiple Myeloma (IMM).

It is a further object of the invention a kit for performing the method of the invention comprising:
amplification and/or detecting and/or quantifying means for at least one marker as defined above;
appropriate reagents.

The kit may also contain instructions for use.

Preferably, the kit of the invention is for use in a method for the prognosis of Symptomatic

Multiple Myeloma (MM) and/or to monitor the response and/or the efficacy of a MM therapy.

It is a further object of the invention a microarray comprising:
solid supporting means;
means able to detect and/or quantify at least one marker as defined above.

Preferably the microarray of the invention is for use in a method for the prognosis of Symptomatic Multiple Myeloma (MM) and/or to monitor the response and/or the efficacy of a MM therapy.

The markers of the invention may be detected and/or measured by any method known to the skilled person in the art.

In the present invention, the term prognosis indicates the possibility: i) to predict that patients affected by myeloma precursor conditions, MGUS and SMM, will evolve to symptomatic MM; ii) to define the risk that patients with symptomatic myeloma (MM) will develop progressive disease during treatment (i.e., fail to respond to therapy, thereby developing refractory myeloma), or will relapse after remission (i.e., patients with complete or very good partial response following treatment but that will relapse, also named relapsing myeloma); iii) to assess the risk of relapse (instead of maintenance) of clinical remission after anti-myeloma treatment.

In the present invention control value refers to:
a value obtained from a sample of patients with no active disease (because of absence of monoclonal plasma cell expansion, as in healthy volunteer (HV) group,
a value obtained from a sample of patients with absence of symptoms in the presence of monoclonal gammopathy as in MGUS,
a value obtained from a sample of patients with remission of myeloma following effective treatment.

A control value may be also a value measured before therapeutic intervention, i.e, anti-myeloma therapy and/or bone marrow transplantation or at different time points during the course of a therapeutic intervention.

The skilled person in the art will know how to select the appropriate control depending on the stage in which the method of the invention is applied and the response desired.

The markers of the invention may be combined in at least 2, 3, 4, 5, 6, 7, 8, 9 10, 11 etc. Any combination may be used to perform the present method. Preferred combinations include any combination of the 25 first markers as indicated in Table 3, any combination of the 20 first markers as indicated in Table 3, combination of the 15 first markers as indicated in Table 3, combination of the 10 first markers as indicated in Table 3, combination of the 5 first markers as indicated in Table 3. Preferably the combinations always include the C3f peptide or fragment thereof. Still preferably the combinations always include the 16 LPC as indicated in Table 4 and 5. One preferred combination includes the detection and/or quantification of C3f peptide or a fragment thereof, Sarcosine (N-Methylglycine), 3-hydroxykynurenine and Pro-hydroxy-pro.

In the present invention detecting and/or quantifying the marker(s) may be performed by any suitable means available in the art and known to the skilled person in the art.

In the present invention the following abbreviations are used: BM, bone marrow Ig, immunoglobulin LPC, lysophosphocholines MGUS, monoclonal gammopathy of undetermined significance MM, multiple myeloma MS, mass spectrometry NMR, nuclear magnetic resonance OOB, out-of-bag OPLS-DA, orthogonal projection to latent structures (or orthogonal partial least squares)—discriminant analysis PC, plasma cells PCA, principal component analysis RF, random forests rs, Spearman coefficient

The invention will be now described by means of non limiting examples in reference to the following figures.

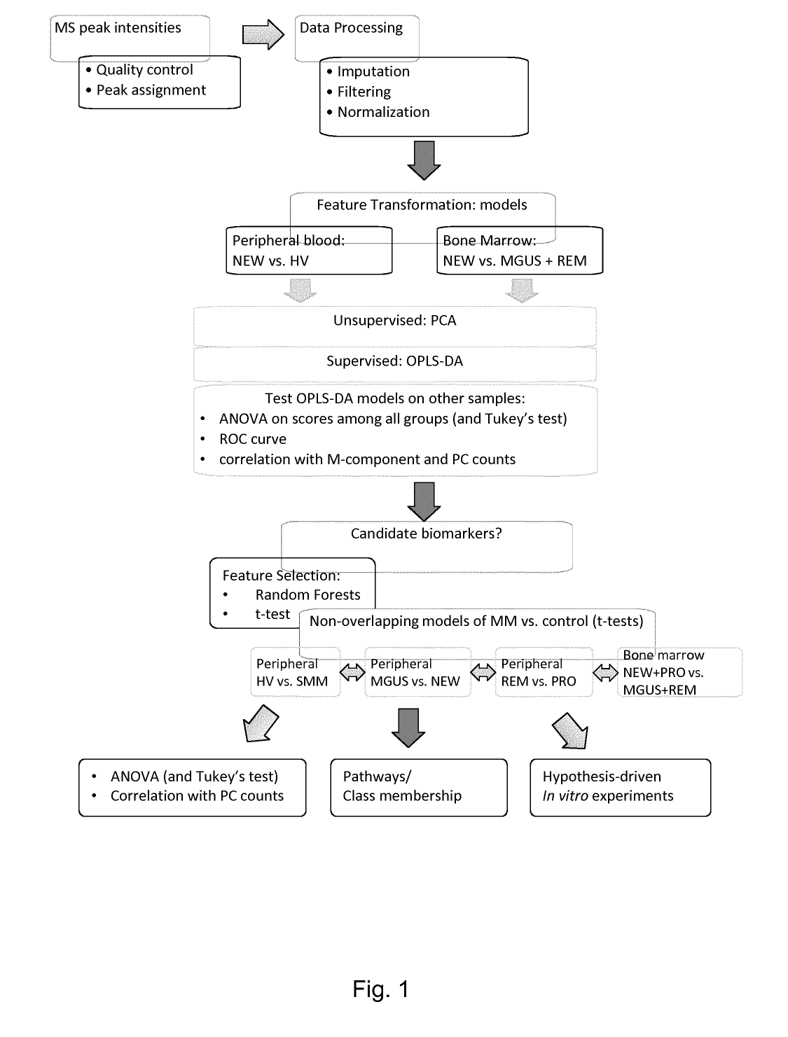

FIG. 1. Experimental workflow

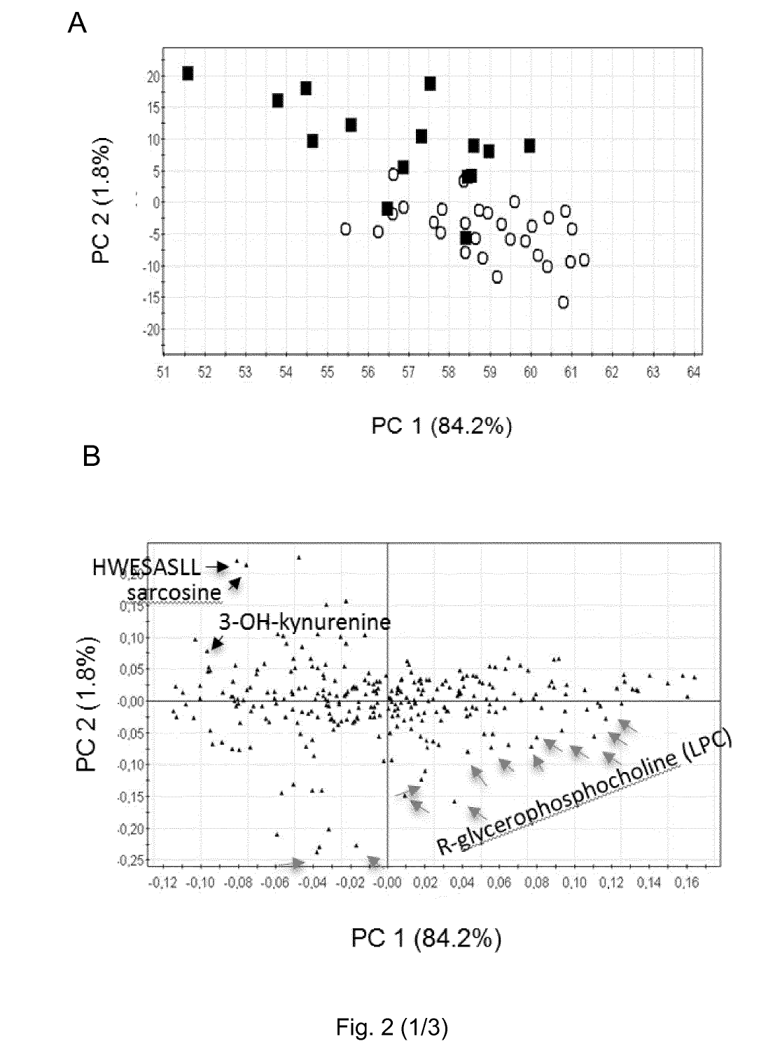

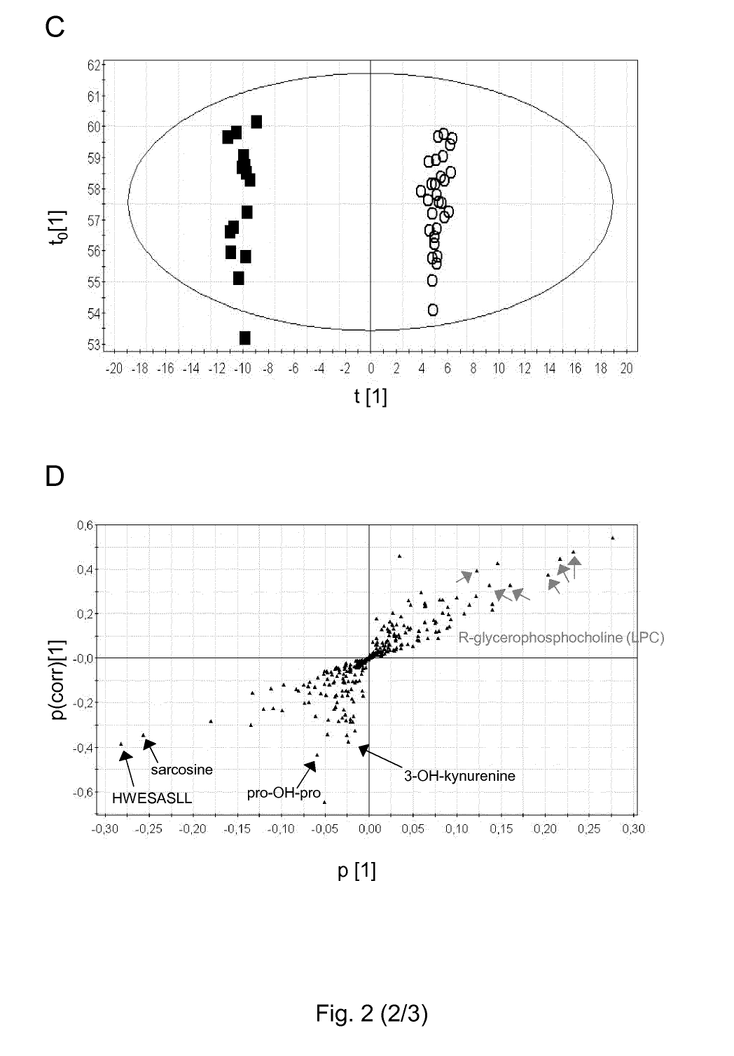

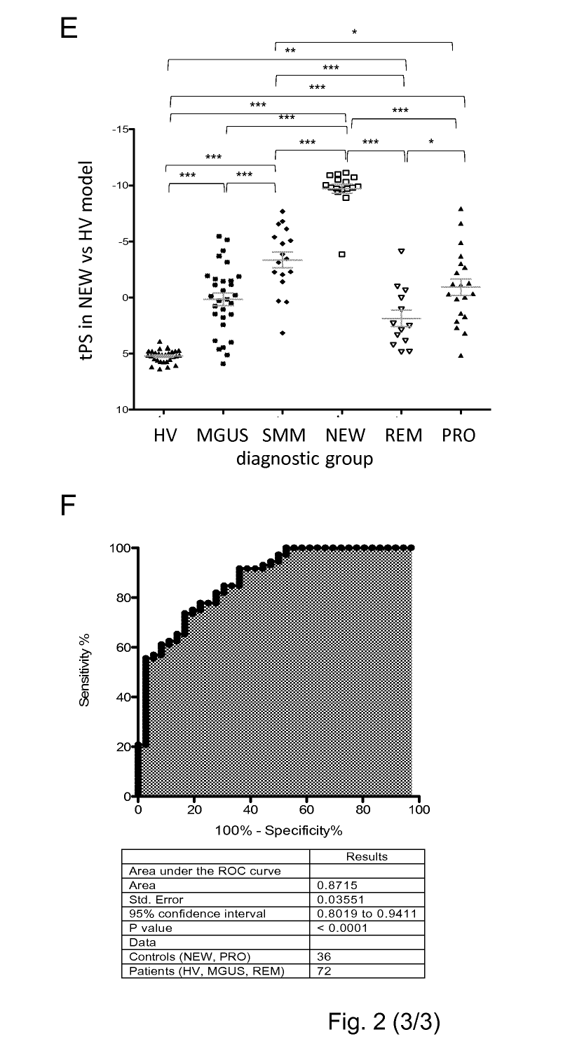

FIG. 2. Metabolic profile analysis of peripheral plasma based on differences between newly diagnosed symptomatic MM patients and healthy controls. A-B) Unsupervised analysis by PCA places samples of the two groups in different regions of the score plot. A) PC1 (84.2% of variability) vs. PC2 (1.8%) score plot, showing as black squares newly diagnosed MM samples (NEW), and as circles healthy controls (HV). B) loading plot for PC1 vs. PC2, highlighting HWESASLL, sarcosine and 3-OH-kynurenine (enriched in NEW samples) and compounds of the glycerophosphocholine class (differing for the R-groups, also known as lysophosphocholines, LPC, gray arrows). C) OPLS-DA score plot, discriminating NEW (black squares, clustering on the left) from HV (circles, on the right). D) OPLS-DA S-plot, black arrows indicating discriminating metabolites, gray arrows indicating individual LPC. E) tPS1 (predicted scores) of training and test samples according to the OPLS-DA model in C and D, with asterisks indicating significance by Tukey's post-test on ANOVA for multiple group comparisons (* p<0.05, ** p<0.01, *** p<0.001). F: Receiver Operating Characteristics (ROC) curve, including NEW and PRO samples as disease (n=35) vs. HV, MGUS and REM samples as controls (n=68), with an area under the curve (AUROC) of 0.8769 and p<0.0001. PRO=relapsing/progressive; REM=remitting.

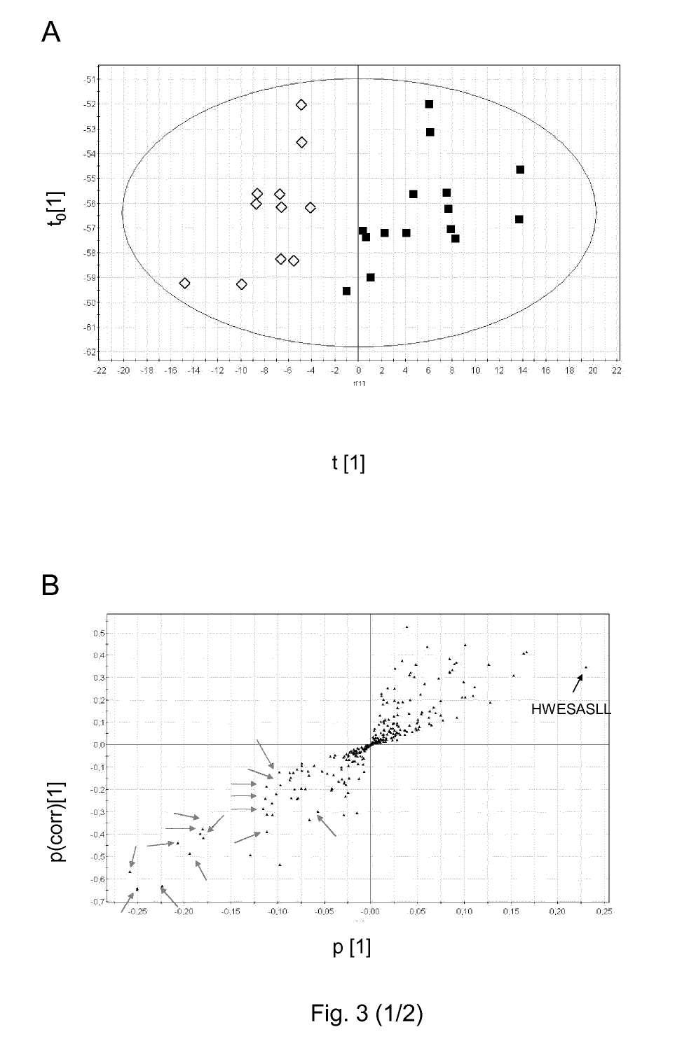

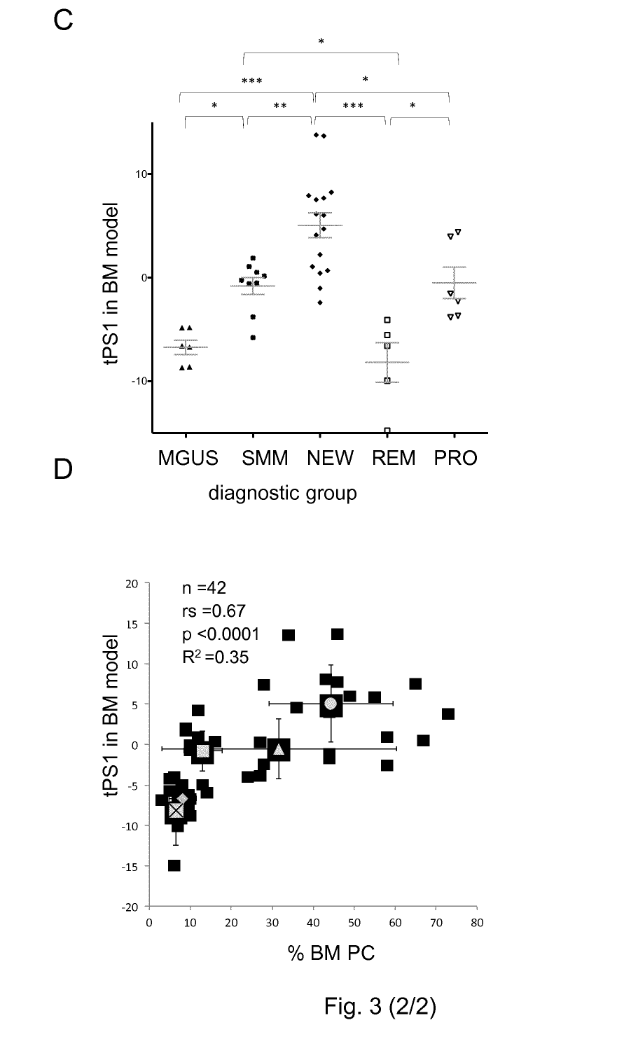

FIG. 3. Bone marrow metabolic fingerprint OPLS-DA model and score. A) OPLS-DA comparing bone marrow samples of newly diagnosed symptomatic myelomas (NEW, black squares) with MGUS+REM samples (white rhombi). B) S-plot, highlighting the contribution of LPC (gray arrows), HWESASLL, sarcosine, 3-OH-kynurenine and pro-OH-proline). C) Scores of training and test samples according to the OPLS-DA model in A and B, with asterisks indicating significance by Tukey's post-test on ANOVA for multiple group comparisons (* p<0.05, ** p<0.01, *** p<0.001). D) Correlation between bone marrow plasma cell count (% BM PC) at the synchronous diagnostic biopsy and the score according to the OPLS-DA model (tPS) in panels A and B (rs 0.61, p<0.001), with dots indicating single patients and gray shapes indicating the median+/−standard deviation (error bars) of the diagnostic groups (crossed square REM, rhombus MGUS, square SMM, triangle PRO and circle NEW).

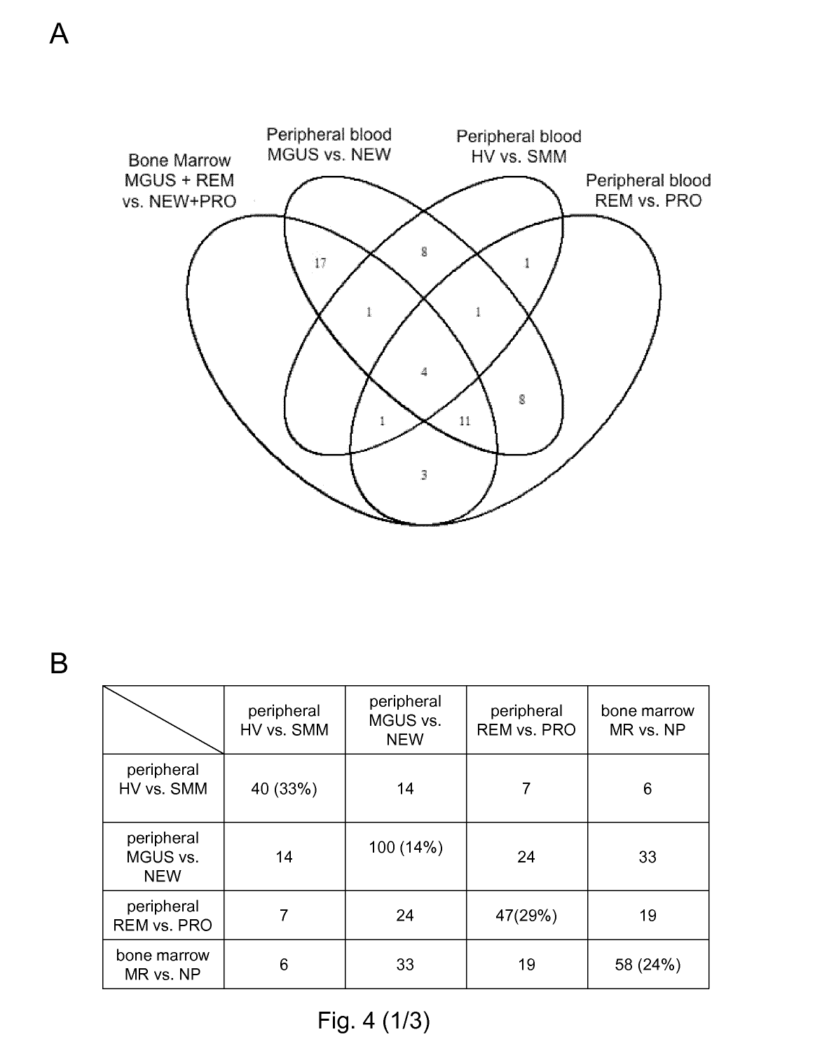

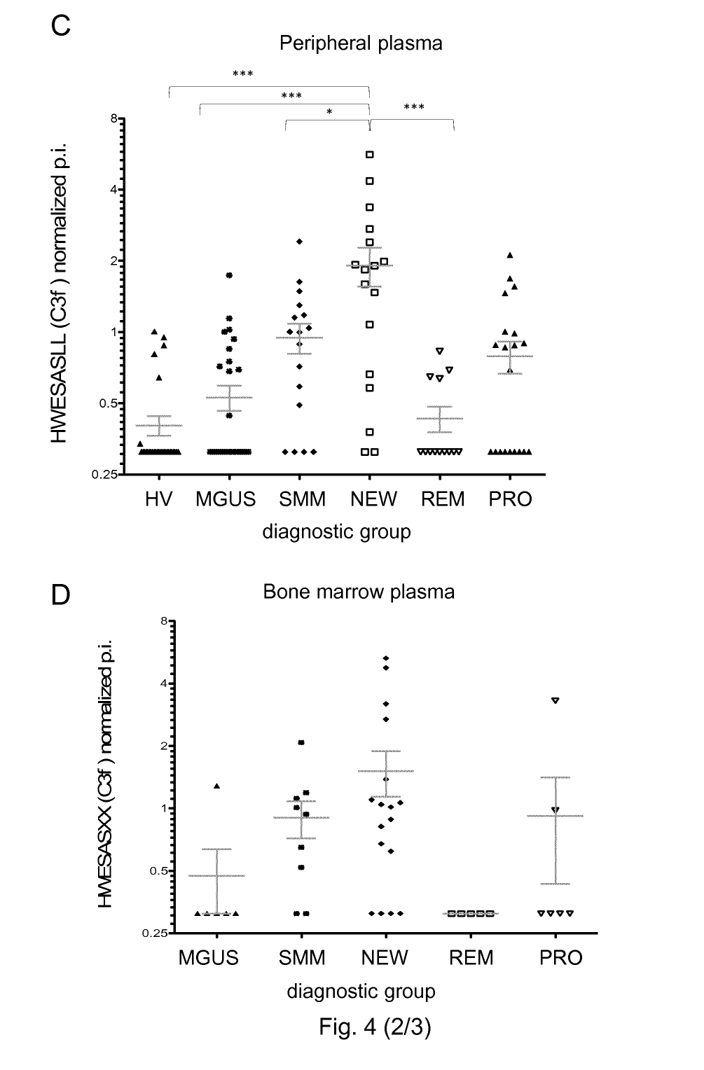

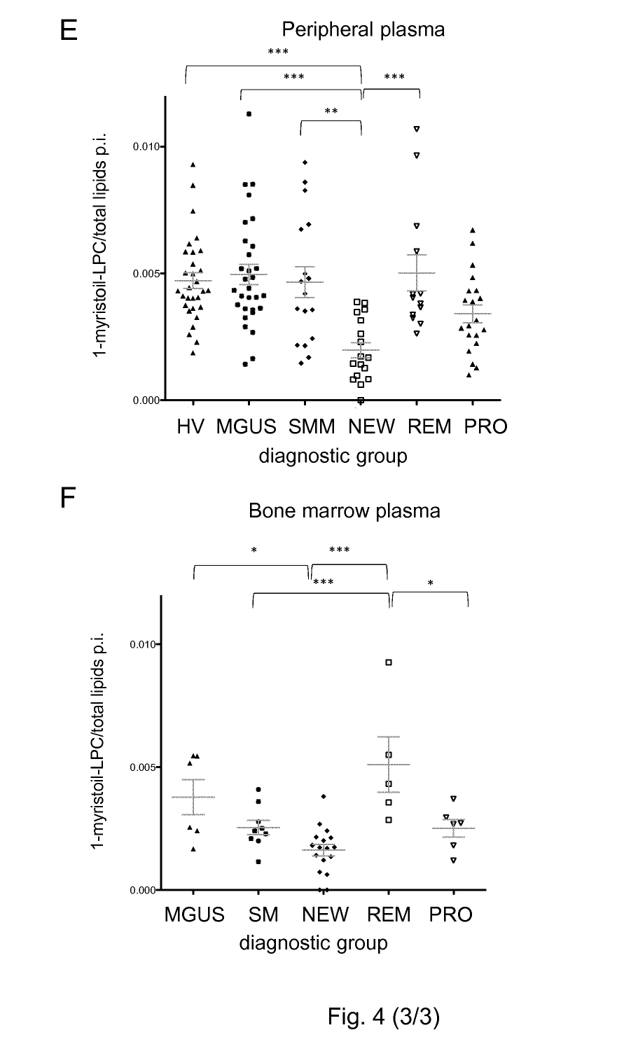

FIG. 4. Selection of individual metabolites as potential markers. A-B) Unpaired t-tests were run for the following non-overlapping groups of samples: bone marrow NEW+PRO vs. MGUS+REM, peripheral NEW vs. MGUS, PRO vs. REM, and HV vs. SMM plasma samples. The identified metabolites with significant differences (p<0.05) are listed in Table 5. The number of shared metabolites with p<0.05 are summarized in the Venn diagram in panel A. The total number of common significant metabolites by any two tests are listed in panel B, the diagonal showing the total number of significant features and false discovery rate (FDR) for each test. C-D) HWESASLL C3f fragment levels in the peripheral (C) and BM (D) plasma (normalized peak intensity). Asterisks highlight statistically significant differences (ANOVA and Tukey's test for multiple comparison analysis) between groups (*p<0.05, ** p<0.001, ***p<0.0001) E-F) Peripheral (E) and BM (F) plasma levels of 1-myristoylglycerophosphocholine decrease in myeloma relative to controls. Values refer to 1-myristoylglycerophosphocholine peak intensity divided by the sum of peak intensities of all lipids (after imputation of missing values and log-scaling). Asterisks highlight statistically significant differences (ANOVA and Tukey's test for multiple comparison analysis) between groups (*p<0.05, ** p<0.001, ***p<0.0001)

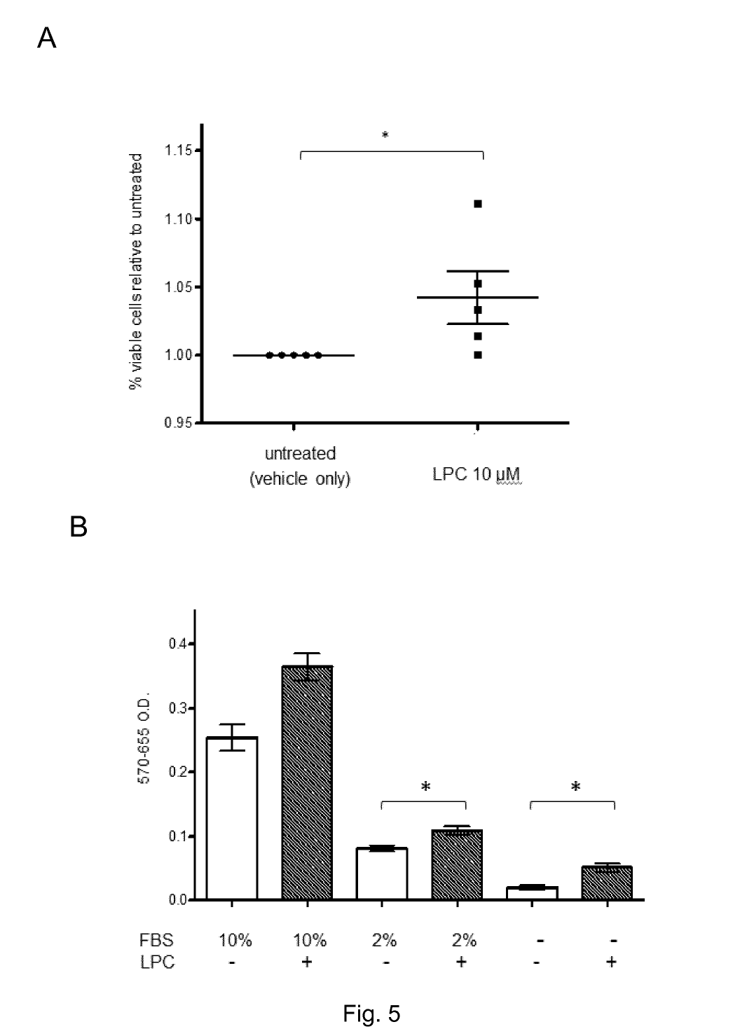

FIG. 5. Effects of LPC supplementation on MM cells viability. A) Viability of myeloma cell lines MM.1S or OPM2 or three samples of myeloma patient-derived CD138 positive cells upon 24 h incubation with vehicle or 10 μM LPC, as fold change of annexinV-propidium iodide double negative cells relative to vehicle alone. B) MTT assay of OPM2 cells upon 72 h supplementation with LPC, one representative experiment (n=6). Y axis: median 570-655 optic density (O.D.); error bars: standard deviation; asterisks: p<0.05 at two-tailed t-test for paired samples.

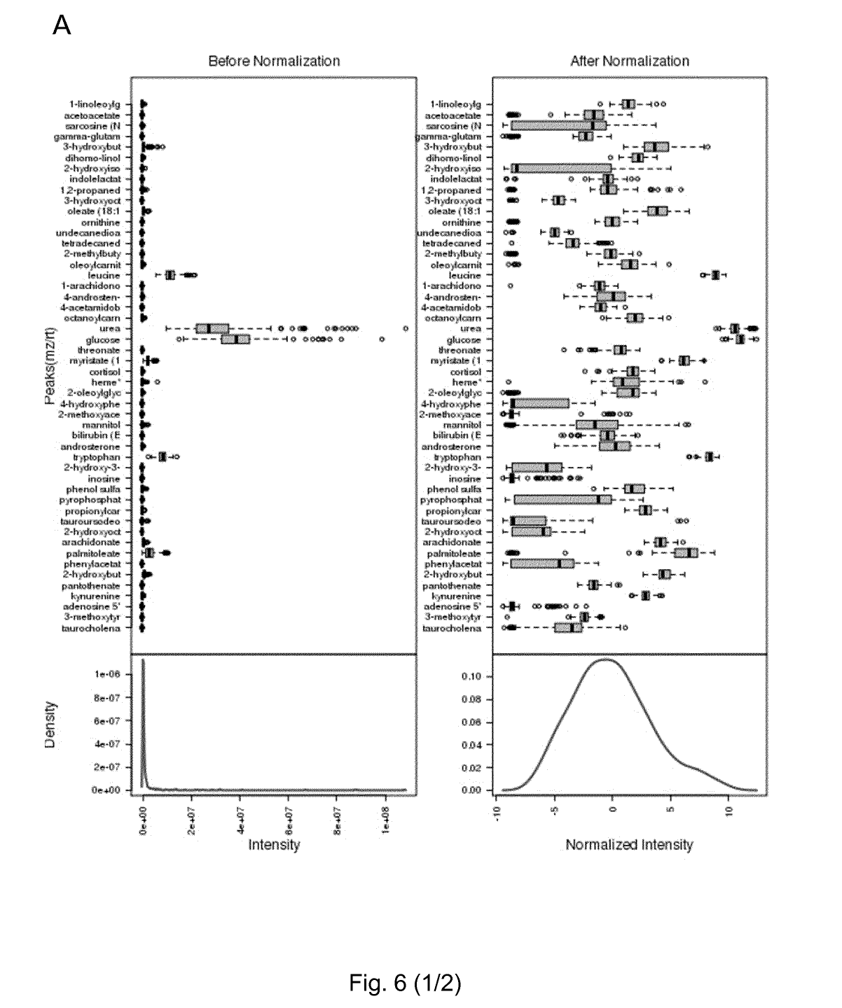

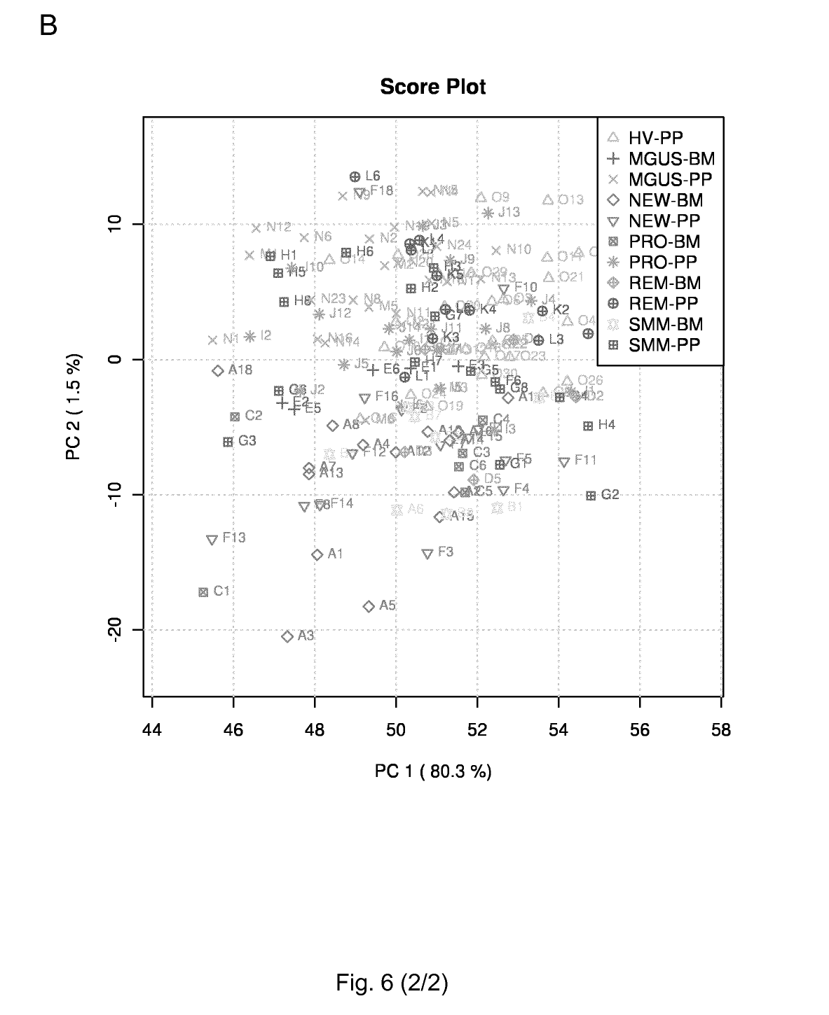

FIG. 6. Data normalization and distribution. A) Summary of the distribution of raw peak intensity data values before and after normalization, showing the concentration distributions of randomly picked individual compounds on top, and overall concentration distribution based on Kernel density estimation in the bottom plots. B) Overall normalized data distribution by PCA, showing no major outliers to drive maximal variability.

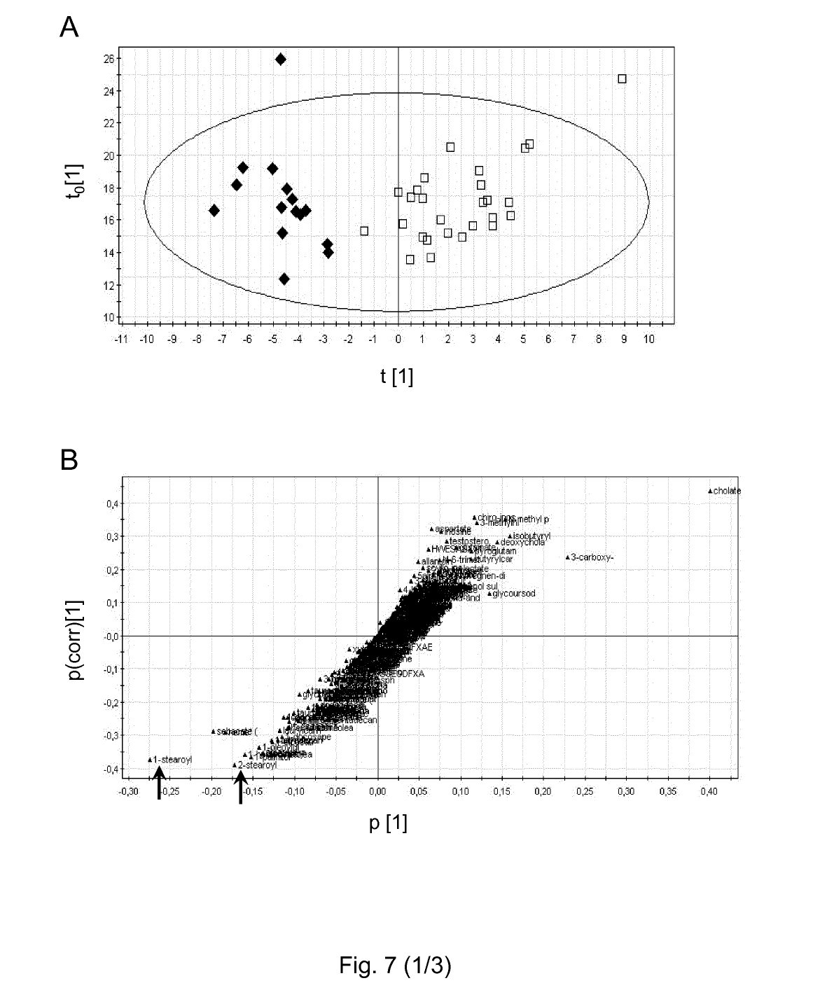

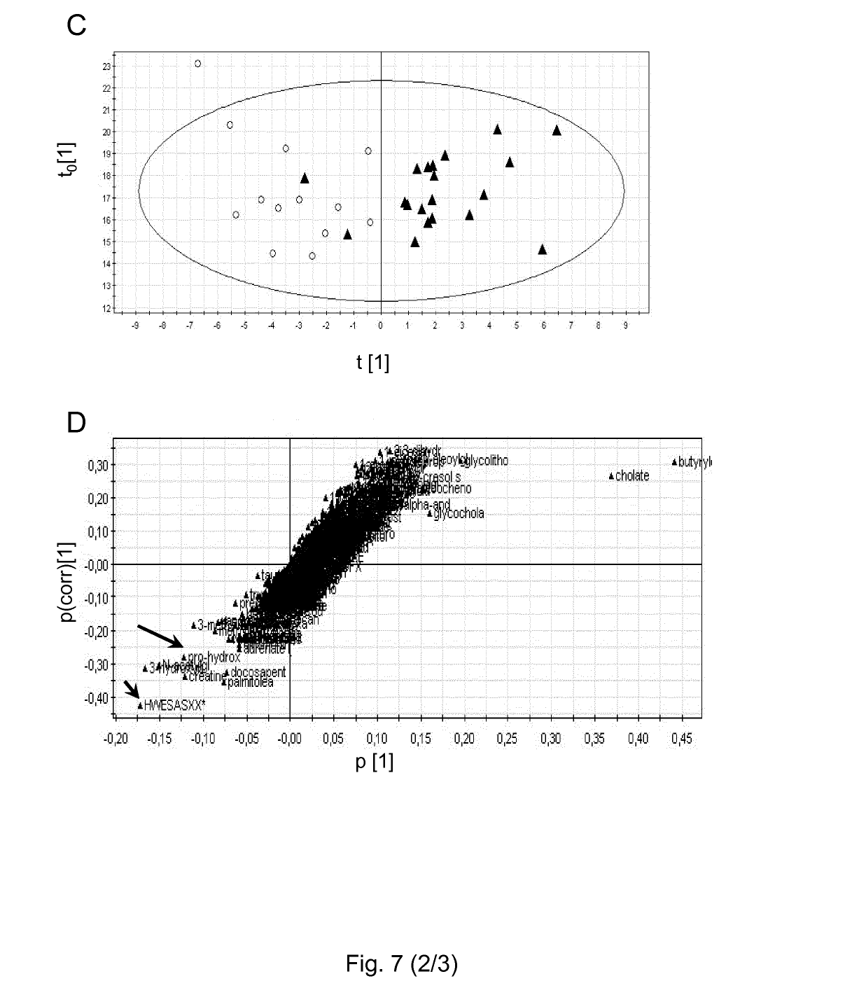

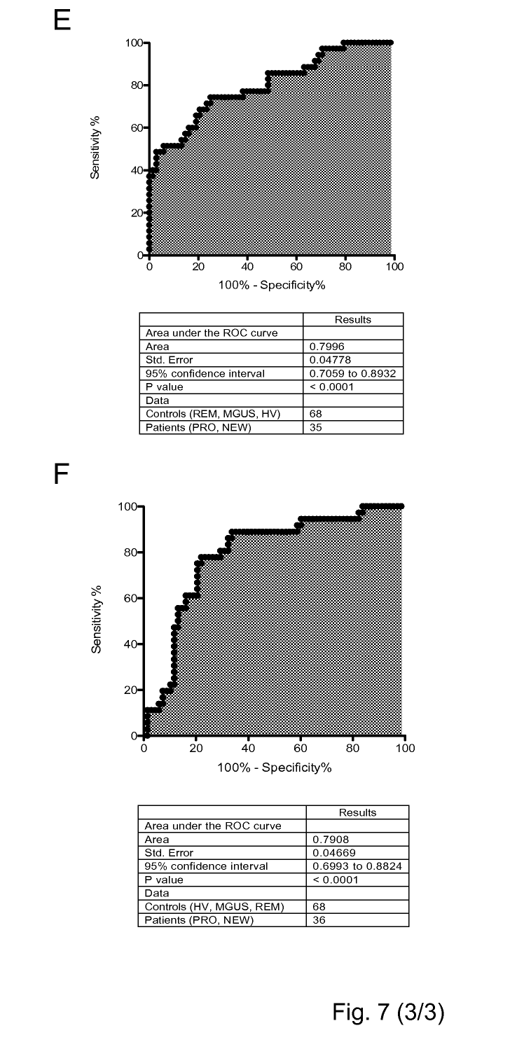

FIG. 7. Alternative training models of disease vs. control by OPLS-DA on peripheral blood profiles. A-B) OPLS-DA model (R2 0.4, Q2 0.15) comparing NEW (newly diagnosed MM, black rhombi) and MGUS (white squares): score plot (A) and S-plot (B). C-D) OPLS-DA model (R2 0.15, Q2 0.05) comparing relapsing/progressive (PRO, black triangles) and remitting (REM, white circles) peripheral blood samples: score plot (C) and S-plot (D). E-F) Receiver Operating Characteristics curve for predicted scores of all disease-free (HV, MGUS, REM) vs. all active disease (PRO and NEW) peripheral blood samples according to the NEW vs. MGUS (in E) or PRO vs. REM (F) models. p[1]: predictive component; t[1]: scores of observations in the predictive component; p(corr)[1]: correlation with the predictive component; to scores of observations on the component orthogonal to the predictive component.

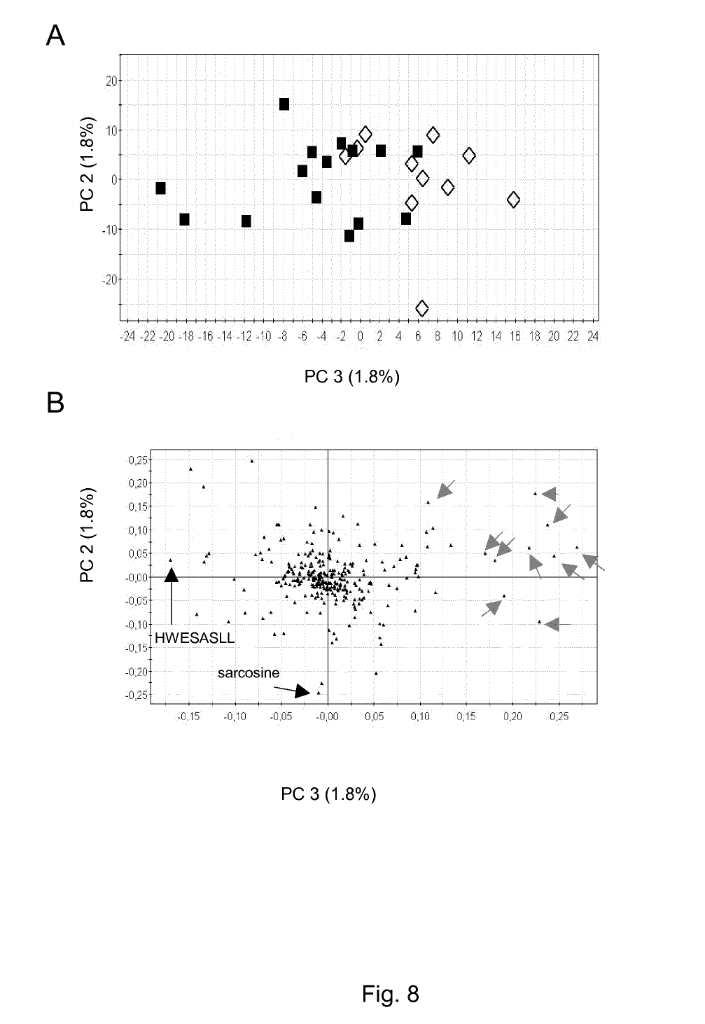

FIG. 8. PCA of bone marrow NEW vs. MGUS+REM metabolic profiling. A) Score plot (NEW in black squares, MGUS and REM in white rhombi) of PC3 (1.8%) vs. PC2 (1.8%). B) Loading plot, black arrow highlighting the contribution of C3f (HWESASLL) and sarcosine, gray arrows indicating LPC.

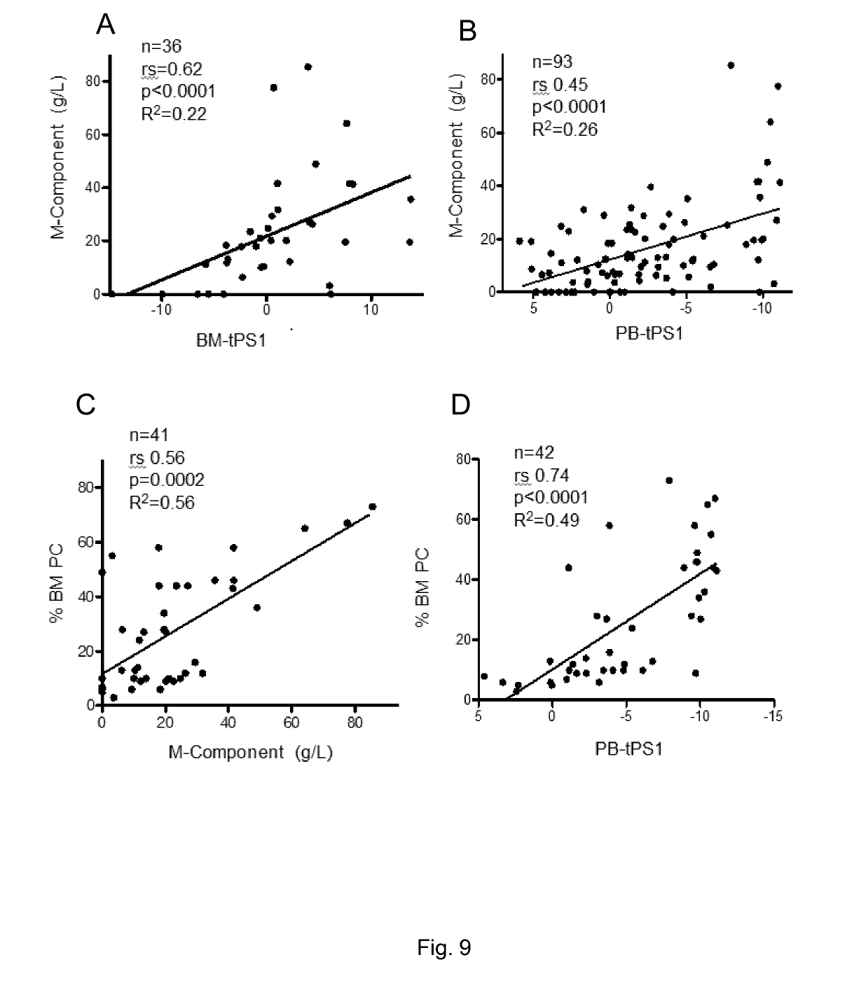

FIG. 9. Correlations of peripheral blood metabolic score (tPS1 based on OPLS-DA of NEW vs. HV differences as in FIG. 2) with M-component levels and BM PC counts. n: number of samples, rs: Spearman correlation coefficient. A) Correlation between BM t predicted score (BM-tPS1, x axis) and M-component (g/l, y axis). B) tPS1 of the peripheral blood (PB-tPS1) correlates with the M-component. C) Correlation between M-component (x axis) and PC count at BM biopsy (% BM PC, y axis). D) Correlation between peripheral blood metabolic score (PB-tPS1, x axis) and PC count at BM biopsy (% BM PC, y axis)

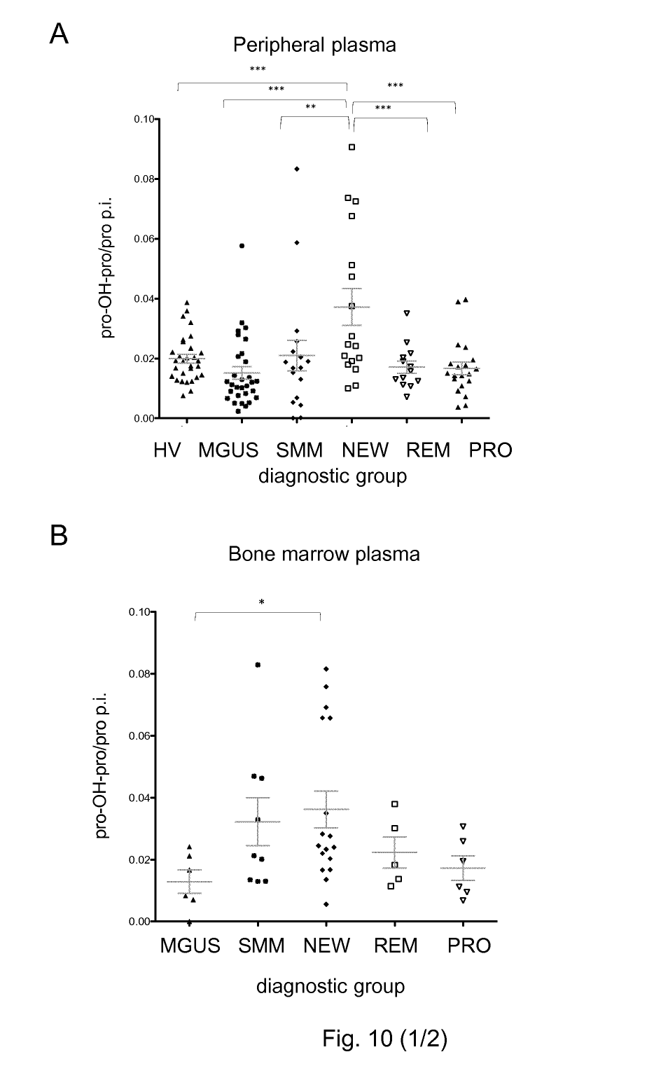

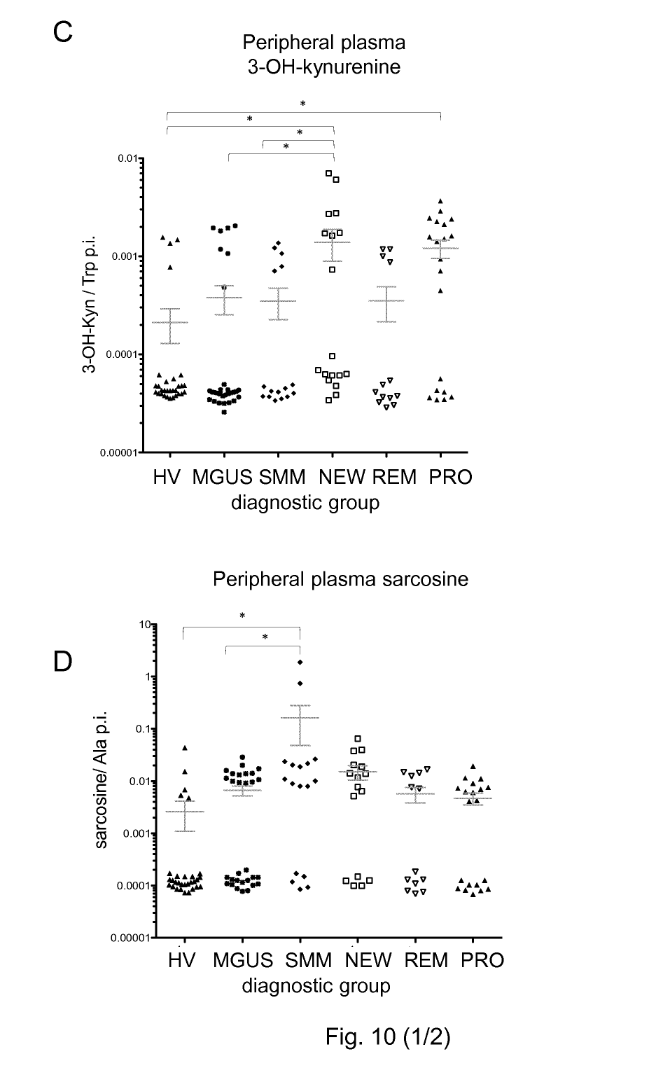

FIG. 10. Aminoacid metabolites associated with multiple myeloma. A-B) pro-hydroxy-proline in peripheral (A) and BM (B) plasma. Peak intensity levels normalized to proline. C) 3-hydroxykynurenine to tryptophan ratio in peripheral blood samples, showing increased levels in NEW samples relative to HV, MGUS or SMM, and high levels in PRO (y axis on log scale). D) Sarcosine levels in the peripheral blood, as peak intensities normalized over alanine (y axis on log scale). Asterisks highlight statistically significant differences (ANOVA, Tukey's test) between groups (*<0.05, **<0.001, ***<0.0001).

### METHODS

### Patients

Upon informed consent subscription, as approved by the institutional review board, 167 samples were obtained from MM or MGUS patients at Ospedale San Raffaele from 2009 to 2011. Age-matched healthy volunteers were also enrolled, upon exclusion of anemia (Hb>12 g/dl), renal dysfunction (serum creatinine<1 mg/dl), gammopathy, clinically evident cancer and ongoing anti-cancer treatments (Table 1).

TABLE 1
Patients characteristics.

| | Samples n. | | Age | Sex | | Comorbidities | | | | | | | | |
|---|---|---|---|---|---|---|---|---|---|---|---|---|---|---|
| Group | PB | BM | median (range) | F | M | C.V. | In | DH | Other Cancer/MDS | Re | CI/AI | En | NP | other |
| HV | 29 | 0 | 66 (39-85) | 20 | 9 | 6 | n | 3 | 1 liver, 1 skin, 1 breast | n | n | n | n | n |
| MGUS | 30 | 6 | 68 (29-85) | 13 | 17 | 7 | 3 | 5 | 1 breast 1 testis | 2 | 3 | 4 | 2 | 2 |
| SMM | 17 | 9 | 66 (45-79) | 9 | 8 | 9 | 2 | 3 | 2 prostate | n | 4 | n | n | n |
| NEW | 16 | 16 | 67 (42-87) | 8 | 8 | 12 | 1 | 2 | 1 prostate | 4 | 1 | 1 | 1 | 2 |
| REM | 13 | 5 | 63 (43-72) | 7 | 6 | 4 | 1 | n | 1 breast | 2 | n | n | 2 | |
| PRO | 20 | 6 | 66 (44-88) | 9 | 11 | 9 | 2 | n | 1 testis, 1 MDS | n | 2 | 1 | n | 2 |

Abbreviations:
C.V., cardiovascular co-morbidities, including hypertension;
In, Chronic Infection (HCV, HBV, tuberculosis);
DH, common metabolic disorders (diabetes or dyslipidemia);
MDS, myelodysplastic syndrome;
Re, respiratory diseases;
CI/AI diseases associated with chronic inflammation or autoimmunity;
En, endocrine disorders;
NP, neurological or psychiatric conditions.
n = none.

### Experimental Design

Samples were classified into 6 groups: HV (healthy individuals), MGUS, SMM (smoldering myeloma), NEW (newly diagnosed, symptomatic MM), REM (in complete response or very good partial response[14] following anti-myeloma therapy, prior to or after bone marrow transplantation), and PRO (relapsing after response, or with progressive[14] disease). Data analysis first addressed differences between peripheral plasma of HV and NEW samples. Following unsupervised principal component analysis (PCA), OPLS-DA models were created to score samples (tPS1) and test inter-group differences, correlations, sensitivity and specificity. Random forests (RF) and ANOVA/t-test were used to select and score individual metabolites of interest. A similar strategy was applied to other disease vs. control pairs. The results of non-redundant pairwise analyses were eventually crossed to obtain a list of candidate biomarkers (FIG. 4A, Table 5).

### Sample Collection, Preparation and Analysis

125 peripheral and 42 bone marrow samples were collected in EDTA-coated vacutainers (BD), immediately transferred in ice and relabeled. Following centrifugation (400 g, 5 min, 4° C.), supernatants were collected with a 22G needle avoiding the upper clumpy layer, filtered (0.22 μm, Millipore), centrifuged (1600 g, 15 min, 4° C.), and stored at −80° C. within 30 minutes of puncture. Metabolic profiling was performed at Metabolon Inc. by UHPLCGC-MS (ultra high performance liquid/gas chromatography and mass spectrometry) as previously described. [11, 12]. Briefly, after extraction with organic and aqueous solvents, each sample was split and analyzed through GC followed by electron impact ionization MS, or UHPLC followed by LQT-FT MS or MS/MS. Quality controls consisted in the chromatographic solvents, a standard of pooled human plasma samples and an internal standard of pooled study samples. Peak assignment and compound identification were obtained through a Metabolon proprietary database of >1,000 compounds and returned as semi-quantitative compound peak intensity tables[7, 11, 12].

### Data Analysis

Data processing included imputation of missing values, data filtering (feature and sample exclusion), and normalization, as described [15-17]. Missing values were imputed as half of the minimum observed peak intensity in the positive samples for the same metabolite using MetaboAnalyst (www.metaboanalyst.ca) [15, 17]. Out of 358 named metabolites, the 69 listed in Table 2 were excluded as possibly related to drug or food intake, or procedures (e.g. fibrinogen fragments upon biopsy).

TABLE 2
Metabolites excluded from analysis

| Metabolite name | Class/Reason for exclusion |
|---|---|
| 1,3,7-trimethylurate | Caffeine metabolite |
| 1,6-anhydroglucose | Cellulose burning metabolite |
| 1,7-dimethylurate | Caffeine metabolite |
| 1-hydroxy-2-naphthalenecarboxylate | Drug (adrenergic |
| | bronchodilator) |
| 1-methylxanthine | Food component |
| 2-hydroxyacetaminophen sulfate* | Acetaminofen metabolite |
| 2-hydroxyhippurate (salicylurate) | Aspirin metabolite |
| 2-methoxyacetaminophen glucuronide* | Acetaminofen metabolite |
| 2-methoxyacetaminophen sulfate* | Acetaminofen metabolite |
| 3-(cystein-S-yl)acetaminophen* | Acetaminofen metabolite |
| 3-hydroxyhippurate | Benzoate metabolism |
| 4-acetamidophenol | Acetaminofen, drug |
| 4-acetaminophen sulfate | Acetaminofen metabolite |
| 4-ethylphenylsulfate | Benzoate metabolism |
| 4-hydroxyhippurate | Benzoate metabolism |
| 4-hydroxynimesulide* | Nimesulide metabolite |
| 4-vinylphenol sulfate | Benzoate metabolism |
| 5-acetylamino-6-amino-3-methyluracil | Xanthine metabolism |
| 7-methylxanthine | Xanthine metabolism |
| ADpSGEGDFXAEGGGVR* | Fibrinogen fragment (altered |
| | in invasive procedures) |
| ADSGEGDFXAEGGGVR* | Fibrinogen fragment (altered |
| | in invasive procedures) |
| Alpha-tocopherol | Vitamin (supplemented in |
| | some patients) |
| Anserine | Food component (enriched in |
| | specific foods) |
| Ascorbate (Vitamin C) | Vitamin (supplemented in |
| | some patients) |
| Atenolol | Drug (β-blocker) |
| Bradykinin | Endogenous peptide susceptible |
| | to alteration by common drugs |
| Bradykinin, des-arg(9) | Endogenous peptide susceptible |
| | to alteration by common drugs |
| Bradykinin, hydroxy-pro(3) | Endogenous peptide susceptible |
| | to alteration by common drugs |
| Caffeine | Food component |
| Carbamazepine 10,11-epoxide* | Carbamazepine metabolite |
| Carbamazepine glucuronide* | Carbamazepine metabolite |
| Carbamazepine* | Drug (anticonvulsant) |
| Cinnamoylglycine | Food component |
| Codeine | Drug (opiate) |
| Cotinine | tobacco metabolism |
| Desmethylnaproxen sulfate* | Naproxene metabolite |
| DSGEGDFXAEGGGVR* | Fibrinogen fragment (altered |
| | in invasive procedures) |
| EDTA | Chelating agent used as |
| | anticoagulant for blood samples |
| Gabapentin | Drug (anticonvulsant, also |
| | used for pain) |
| Gamma-CEHC | Vitamin E metabolite, altered |
| | in smokers |
| Glycolate (hydroxyacetate) | Employed in cosmetic and |
| | pharmaceutical preparations |
| Hippurate | Benzoate metabolism |
| Homostachydrine* | Food component |
| Hydrochlorothiazide | Drug (diuretic) |
| Lidocaine | Drug (local anesthetic, |
| | antiarrhythmic agent) |
| Metoprolol | Drug (β-blocker) |
| Metoprolol acid metabolite* | Metoprolol metabolite |
| N-ethylglycinexylidide* | Lidocaine metabolite |
| Naproxen | Drug (NSAID) |
| Nimesulide* | Drug (NSAID) |
| Ofloxacin | Drug (fluoroquinolone, |
| | antibiotic) |
| Omeprazole | Drug (proton pump inhibitor) |
| p-acetamidophenylglucuronide | Acetaminofen metabolite |
| Pantoprazole | Drug (proton pump inhibitor) |
| Paraxanthine | Xanthine metabolism |
| Pipecolate | Drug (rapamycin, |
| | immunosuppressant) |
| Pivaloylcarnitine | Isovalerylglycine isomer |
| | (HMDB00678) deriving from |
| | pivalate-generating antibiotics |
| Quinate | Food component (coffee, |
| | carrots, tobacco . . . ) |
| Ranitidine | Drug (H2-receptor blocker) |
| Saccharin | Food component |
| Salicylate | Drug (Aspirin, NSAID) |
| salicyluric glucuronide* | Aspirin metabolite |
| Stachydrine | Food/herbal extracts |
| | component |
| Tartarate | Food component |
| Theobromine | Food component or drug |
| | (DB01412) |
| Theophylline | Food component, drug |
| | (DB00277) |
| Thymol sulfate | Herb extract |
| Ticlopidine* | Drug (antiplatelet) |
| Topiramate | Drug (anticonvulsant) |

Samples from patients with hepatic dysfunction (bilirubin>1 mg/dl, plus history of chronic liver disease or elevated serum hepatic enzymes) were excluded from PCA and the training sets of OPLS-DA, to be only reintroduced in the test series. Peak intensities were normalized by median-centering and log-scaling (log 2), and verified to have a suitable distribution (FIG. 6A-B, Kernel distribution and PCA score plot) for multivariate analysis [15].

The SIMCA-P+software (Umetrics) was used for PCA and OPLS-DA to study inter-group differences and create models based on sample training sets. The t1 score defining the OPLS-DA model was then predicted for the other myeloma samples by including them as a prediction set (tPS1). MetaboAnalyst was also used for random forests (RF), PCA, Spearman correlation rank, t-test and false discovery rate (FDR) determination. Graph Pad Prism Software was used for the other statistics.

### In Vitro Experiments

Myeloma cell lines (OPM2 and MM.1S) were cultured in RPMI1640 media (Gibco), supplemented with glutamax (1 mM), penicillin (100 U/ml), and streptomycin (100 μg/ml). Primary MM cells were obtained by CD138 positive immunomagnetic selection (Miltenyi) from bone marrow mononuclear cells. CD138⁺ cells were cultured in 10% FBS and IL-6 (2 ng/ml). Apoptosis was detected by AnnexinV-PI (BD) cytometry (AccuriC6 cytometer, analyzed with FCS-express). For MTT assays, OPM2 cells were cultured with or without 10 μM LPC and FCS, incubated with 5 mg/ml MTT (Sigma), dissolved with DMSO and measured for ABS at 570-655 nm with an ELISA reader (Biorad).

### Results

To investigate the metabolic correlates of MM development and progression, the authors collected blood samples from patients newly diagnosed with MM (NEW, n=16), with relapsing or progressive disease (PRO, n=20), in clinical remission (REM, n=13), with MGUS (n=30), with SMM (SMM, n=17) and from age-matched healthy volunteers (HV, n=29). In 42 cases, the authors obtained synchronous BM aspirates, collected for diagnostic purpose. The general experimental workflow and patient characteristics are respectively summarized in FIG. 1 and Table 1. Metabolic profiles were analyzed by UHPLCGC-MS, resulting in 359 named metabolites, 284 of which were considered endogenous, and included in statistical analyses (see Methods for further details on experimental design and analytic strategies).

Multivariate Analysis of Metabolic Footprinting in Peripheral Plasma Discriminates Myeloma Cases from Healthy Controls

To generate a first metabolic model the authors used peripheral blood from the two most distant conditions: newly diagnosed untreated symptomatic patients (NEW), and age-matched healthy volunteers (HV). Principal Component Analysis (PCA) shows NEW and HV samples to fall in separate areas of the score plot of the two principal components (PC1 and PC2, FIG. 2A-B). The exo-metabolome being the result of processes with high inter-individual variability (such as diet, sex and genetic background), PCA, an unsupervised analysis focusing on maximal variance and disregarding class membership of samples, often fails to discriminate patients and controls [16, 18]. In myeloma, a difference between NEW and HV already emerges in the first two components, indicating robust metabolic differences between the two groups. With a supervised approach, OPLS-DA [19] separates the two groups into two well-defined clusters based on metabolic profile (R2Y 0.8, Q2 0.5, RX1=0.013 FIG. 2C-D). The authors then tested the resulting model in samples derived from the other groups. Remarkably, when metabolic profiles of the other groups were analyzed, their scores (tPS1) followed the predicted trend for disease progression (panel E), accurately capturing differences that were not originally included in the training model. In particular, SMM patients scored significantly higher than healthy or MGUS (p<0.0001) and lower than NEW (p<0.0001), and MGUS individuals scored lower than NEW (p<0.0001) but higher than HV (p<0.0001), while REM patients were significantly lower than both NEW (p<0.0001) and PRO (p<0.05). In all, a receiver operating characteristic (ROC) curve comprising patients with (PRO and NEW) and without (HV, MGUS and REM) active disease revealed good sensitivity and specificity of the metabolic score (FIG. 1F, AUROC 0.87, p<0.0001).

As shown in FIG. 7, different training models generated comparing NEW with MGUS (FIG. 7A-B) and REM with PRO (FIG. 7C-D), also yielded good separation by OPLS-DA and, when tested on the entire dataset, produced significant areas under the ROC curve (p<0.0001, FIG. 7E-F).

Feature selecting methods, such as Random Forests (RF)[20], with out-of-bag (OOB) error of 0.109, identified a small set of metabolites contributing to the separation between NEW and HV, which remained significant after multiple testing correction (Table 3, FDR<15%). The 25 highest-ranking features of RF (p<0.0001 FDR<1%) included 9 lysophosphocholines (LPC), concordantly lower in MM than HV samples, and the increase of C3f peptide HWESASLL, creatinine, pro-hydroxy-proline, 3-hydroxykynurenine, and sarcosine (Table S2). Attesting to consistency, these same metabolites contributed to the PCA and OPLS-DA loadings in the direction of inter-group separation (FIG. 2 B,D).

TABLE 3
Complete list of selected features in NEW vs. HV analyses. List of selected metabolites,
crossing the results of Random Forests (with Mean Decreased Accuracy and ranking), OPLS-
DA (p [1] score on loading), t-test (p value and False Discovery Rate), and increase in newly
diagnosed MM (upward arrows) or decrease (downward arrows) relative to healthy controls
(HV).

| | | | OPLS- | | | |
|---|---|---|---|---|---|---|
| | | | DA | | | NEW |
| | Random Forest | | Loading | t test | | vs. |
| Metabolite name | MDA | Rank | P[1] | P | FDR | HV |
| 1-arachidonoylglycerophosphocholine | 1.31E−02 | 1 | −0.120 | 2.02E−08 | 2.68E−06 | ↓ |
| HWESASXX (HWESALL, C3F) | 1.09E−02 | 2 | 0.263 | 3.85E−09 | 1.12E−06 | ↑ |
| 1-myristoylglycerophosphocholine | 1.04E−02 | 3 | −0.146 | 2.78E−08 | 2.68E−06 | ↓ |
| 2-palmitoylglycerophosphocholine | 1.01E−02 | 4 | −0.134 | 3.79E−08 | 2.74E−06 | ↓ |
| 1-linoleoylglycerophosphocholine | 9.25E−03 | 5 | −0.100 | 5.65E−07 | 2.34E−05 | ↓ |
| 1-eicosatrienoylglycerophosphocholine | 6.98E−03 | 6 | −0.122 | 8.87E−08 | 5.14E−06 | ↓ |
| Creatinine | 6.18E−03 | 7 | 0.041 | 2.49E−04 | 3.63E−03 | ↑ |
| Glutaroyl carnitine | 5.78E−03 | 8 | 0.081 | 7.72E−06 | 2.24E−04 | ↑ |
| 2-linoleoylglycerophosphocholine* | 5.17E−03 | 9 | −0.129 | 5.26E−05 | 1.09E−03 | ↓ |
| N-(2-furoyl)glycine | 5.00E−03 | 10 | −0.155 | 2.03E−07 | 9.82E−06 | ↓ |
| 1-palmitoleoyl-glycerophosphocholine* | 4.79E−03 | 11 | −0.109 | 1.45E−05 | 3.51E−04 | ↓ |
| Citrate | 3.68E−03 | 12 | −0.049 | 1.52E−02 | 7.10E−02 | ↓ |
| Carnitine | 3.37E−03 | 13 | 0.038 | 6.35E−06 | 2.04E−04 | ↑ |
| Sarcosine (N-Methylglycine) | 3.25E−03 | 14 | 0.072 | 1.12E−04 | 1.95E−03 | ↑ |
| 3-hydroxykynurenine | 3.19E−03 | 15 | 0.089 | 6.86E−03 | 4.56E−02 | ↑ |
| Xanthosine | 3.11E−03 | 16 | 0.045 | 7.09E−04 | 7.91E−03 | ↑ |
| 1-docosahexaenoyl- | 3.10E−03 | 17 | −0.101 | 1.83E−04 | 2.95E−03 | ↓ |
| glycerophosphocholine | | | | | | |
| Testosterone sulfate | 3.05E−03 | 18 | 0.023 | 6.05E−03 | 4.18E−02 | ↑ |
| 1-palmitoylglycerophosphocholine | 3.02E−03 | 19 | −0.081 | 4.97E−06 | 1.80E−04 | ↓ |
| Glycerol 3-phosphate (G3P) | 2.60E−03 | 20 | −0.096 | 4.11E−04 | 5.42E−03 | ↓ |
| Acetylcarnitine | 2.53E−03 | 21 | 0.074 | 1.17E−05 | 3.09E−04 | ↑ |
| N1-methyladenosine | 2.32E−03 | 22 | 0.016 | 1.13E−03 | 1.13E−02 | ↑ |
| Pro-hydroxy-pro | 2.30E−03 | 23 | 0.134 | 1.68E−05 | 3.74E−04 | ↑ |
| Urate | 2.25E−03 | 24 | 0.028 | 2.51E−04 | 3.63E−03 | ↑ |
| 7-alpha-hydroxy-3-oxo-4-cholestenoate | 2.06E−03 | 25 | 0.026 | 8.55E−04 | 9.18E−03 | ↑ |
| (7-Hoca) | | | | | | |
| Cysteine | 1.87E−03 | 27 | −0.031 | 4.68E−02 | 1.51E−01 | ↓ |
| 2-hydroxybutyrate (AHB) | 1.86E−03 | 28 | 0.074 | 1.07E−03 | 1.11E−02 | ↑ |
| Cortisol | 1.54E−03 | 30 | 0.046 | 1.14E−02 | 6.24E−02 | ↑ |
| 1-oleoylglycerophosphoethanolamine | 1.44E−03 | 31 | −0.042 | 1.16E−02 | 6.24E−02 | ↓ |
| Butyrylcarnitine | 1.30E−03 | 34 | 0.042 | 8.52E−03 | 5.15E−02 | ↑ |
| Pyridoxate | 1.23E−03 | 35 | −0.037 | 2.23E−02 | 9.19E−02 | ↓ |
| Pseudouridine | 1.20E−03 | 36 | 0.057 | 4.75E−04 | 5.51E−03 | ↑ |
| 1-stearoylglycerophosphocholine | 1.16E−03 | 37 | −0.155 | 2.13E−03 | 1.87E−02 | ↓ |
| Palmitoyl sphingomyelin | 1.15E−03 | 38 | −0.062 | 1.34E−03 | 1.30E−02 | ↓ |
| 1-oleoylglycerophosphocholine | 1.14E−03 | 39 | −0.112 | 7.75E−05 | 1.50E−03 | ↓ |
| Creatine | 1.14E−03 | 40 | 0.126 | 4.49E−04 | 5.51E−03 | ↑ |
| 1-docosapentaenoyl- | 1.05E−03 | 41 | −0.101 | 4.00E−04 | 5.42E−03 | ↓ |
| glycerophosphocholine | | | | | | |
| Gamma-glutamylphenylalanine | 1.00E−03 | 42 | 0.020 | 5.96E−03 | 4.18E−02 | ↑ |
| Catechol sulfate | 9.77E−04 | 43 | −0.090 | 4.63E−04 | 5.51E−03 | ↓ |
| 13-HODE + 9-HODE | 8.75E−04 | 44 | −0.063 | 1.49E−03 | 1.40E−02 | ↓ |
| Hexanoylcarnitine | 8.75E−04 | 45 | 0.099 | 2.60E−03 | 2.22E−02 | ↑ |
| 2-hydroxypalmitate | 8.59E−04 | 47 | 0.005 | 2.73E−02 | 1.00E−01 | ↑ |
| Indolepropionate | 7.46E−04 | 49 | −0.097 | 6.92E−03 | 4.56E−02 | ↓ |
| Oleoylcarnitine | 7.22E−04 | 50 | −0.108 | 3.51E−03 | 2.78E−02 | ↓ |
| Succinate | 6.84E−04 | 52 | −0.053 | 3.55E−03 | 2.78E−02 | ↓ |
| Tyrosine | 6.83E−04 | 53 | −0.039 | 1.57E−02 | 7.13E−02 | ↓ |
| Levulinate (4-oxovalerate) | 6.57E−04 | 55 | −0.045 | 2.28E−02 | 9.19E−02 | ↓ |
| Adenosine 5′-monophosphate (AMP) | 6.12E−04 | 58 | −0.077 | 9.62E−03 | 5.58E−02 | ↓ |
| Pyroglutamine | 6.10E−04 | 59 | 0.055 | 1.09E−02 | 6.08E−02 | ↑ |
| Pregn steroid monosulfate | 5.26E−04 | 62 | 0.014 | 2.26E−02 | 9.19E−02 | ↑ |
| Alpha-hydroxyisovalerate | 4.79E−04 | 65 | 0.037 | 1.34E−02 | 6.58E−02 | ↑ |
| Andro steroid monosulfate 2 | 4.77E−04 | 66 | 0.052 | 9.03E−03 | 5.35E−02 | ↑ |
| 3-methylhistidine | 4.26E−04 | 70 | 0.213 | 3.04E−03 | 2.51E−02 | ↑ |
| Lactate | 3.82E−04 | 72 | 0.027 | 8.43E−03 | 5.15E−02 | ↑ |
| Caprate (10:0) | 3.72E−04 | 74 | −0.044 | 4.95E−02 | 1.55E−01 | ↓ |
| 3-hydroxyisobutyrate | 3.71E−04 | 75 | 0.032 | 2.61E−02 | 9.95E−02 | ↑ |
| Scyllo-inositol | 3.46E−04 | 78 | 0.040 | 1.35E−02 | 6.58E−02 | ↑ |
| N-acetyl-beta-alanine | 3.30E−04 | 83 | 0.065 | 1.61E−02 | 7.18E−02 | ↑ |
| 2-aminobutyrate | 3.24E−04 | 85 | 0.025 | 1.28E−02 | 6.58E−02 | ↑ |
| Phenyllactate (PLA) | 3.06E−04 | 89 | 0.036 | 2.16E−02 | 9.08E−02 | ↑ |
| Heptanoate (7:0) | 2.92E−04 | 93 | −0.048 | 1.36E−02 | 6.58E−02 | ↓ |
| Beta-hydroxyisovalerate | 2.91E−04 | 95 | 0.031 | 1.98E−02 | 8.56E−02 | ↑ |
| 3-methoxytyrosine | 2.85E−04 | 96 | 0.029 | 1.02E−02 | 5.79E−02 | ↑ |
| Deoxycarnitine | 2.64E−04 | 99 | 0.044 | 1.14E−04 | 1.95E−03 | ↑ |
| 1-palmitoylplasmenylethanolamine | 2.39E−04 | 106 | −0.050 | 2.64E−02 | 9.95E−02 | ↓ |
| 3-methyl-2-oxobutyrate | 2.10E−04 | 115 | 0.012 | 7.28E−03 | 4.69E−02 | ↑ |
| 3-phenylpropionate (hydrocinnamate) | 2.09E−04 | 116 | −0.162 | 1.98E−03 | 1.79E−02 | ↓ |
| Propionylcarnitine | 2.01E−04 | 119 | 0.072 | 5.72E−03 | 4.15E−02 | ↑ |
| Pelargonate (9:0) | 1.98E−04 | 120 | −0.047 | 1.48E−02 | 7.02E−02 | ↓ |
| Tryptophan | 1.25E−04 | 133 | −0.032 | 3.93E−02 | 1.33E−01 | ↓ |
| 2-stearoylglycerophosphocholine | 1.18E−04 | 136 | −0.097 | 4.98E−02 | 1.55E−01 | ↓ |
| Pregnenolone sulfate | 9.15E−05 | 146 | 0.027 | 4.45E−03 | 3.40E−02 | ↑ |
| Phosphate | 8.46E−05 | 147 | −0.034 | 2.09E−02 | 8.90E−02 | ↓ |
| N-acetylmethionine | 6.44E−05 | 160 | 0.040 | 1.88E−02 | 8.27E−02 | ↑ |
| Caprylate (8:0) | 5.14E−05 | 169 | −0.047 | 2.68E−02 | 9.95E−02 | ↓ |
| N-formylmethionine | 4.71E−05 | 173 | 0.071 | 8.21E−03 | 5.15E−02 | ↑ |
| Cyclo(leu-pro) | 2.30E−05 | 182 | −0.073 | 1.22E−02 | 6.42E−02 | ↓ |
| 1-heptadecanoylglycerophosphocholine | 1.59E−05 | 185 | −0.117 | 1.56E−02 | 7.13E−02 | ↓ |
| Pregnen-diol disulfate | 4.17E−06 | 191 | 0.079 | 4.62E−02 | 1.51E−01 | ↑ |
| Acetylphosphate | −6.15E−06 | 198 | −0.034 | 4.67E−02 | 1.51E−01 | ↓ |
| Taurochenodeoxycholate | −1.93E−05 | 203 | 0.138 | 3.10E−02 | 1.10E−01 | ↑ |
| Arginine | −4.69E−05 | 210 | −0.037 | 4.69E−02 | 1.51E−01 | ↓ |
| Cholesterol | −4.77E−05 | 211 | −0.038 | 3.90E−02 | 1.33E−01 | ↓ |
| C-glycosyltryptophan | −5.19E−05 | 212 | 0.048 | 2.48E−02 | 9.83E−02 | ↑ |
| 4-androsten-3beta. 17beta-diol disulfate 1 | −6.43E−05 | 219 | 0.068 | 3.00E−02 | 1.07E−01 | ↑ |
| N-methyl proline | −9.31E−05 | 226 | −0.096 | 3.53E−02 | 1.22E−01 | ↓ |
| Stearoyl sphingomyelin | −1.13E−04 | 232 | −0.051 | 2.95E−02 | 1.07E−01 | ↓ |
| Mannose | −1.68E−04 | 249 | 0.042 | 2.60E−02 | 9.95E−02 | ↑ |
| 21-hydroxypregnenolone disulfate | −2.13E−04 | 260 | 0.002 | 2.65E−02 | 9.95E−02 | ↑ |
| 1-eicosadienoylglycerophosphocholine | −2.47E−04 | 271 | −0.143 | 5.30E−03 | 3.94E−02 | ↓ |

The Bone Marrow Plasma Metabolome Discriminates Active Myeloma from MGUS and Remitting Disease

As the prime site of myeloma localization[8], the authors tested whether the BM displays cancer-associated metabolic alterations. Since only patients undergoing diagnostic biopsy and aspirate were sampled, the authors combined BM plasma samples from MGUS and REM groups (MGUS+REM) as the closest surrogate to a disease-free condition, and compared them with NEW. The 2 groups were successfully discriminated by OPLS-DA (FIG. 3A-B, R2X 1.6%, R2Y 0.75, Q2 0.23) and RF (OOB error 0.296, features with t-test in Table 4), and separated along PC3 (PCA in FIG. 8). When the resulting model was tested in all groups, relapsing and progressive myelomas (PRO) revealed a significantly higher score than REM (p<0.05); SMM scored lower than NEW (p<0.01) and higher than REM (p<0.05) or MGUS (p<0.05) (FIG. 3C). Importantly, the tPS1 metabolic score of bone marrow plasma was found to correlate with PC counts in the synchronous diagnostic biopsy (rs 0.67, p<0.001, R² 0.35) (FIG. 3D). Interestingly, the metabolites emerging as responsible for discriminating NEW from MGUS+REM (FIG. 3B, 8B, Table 4) included most LPC and the HWESASLL peptide.

TABLE 4
Relevant features in bone marrow NEW vs. MGUS + REM comparison. Features
selected by Random Forests (RF) for comparison of BM profiles of newly diagnosed myelomas
(NEW) vs. MGUS + REM samples, showing RF mean decreased accuracy (MDR) and ranking,
and significant t-test results with a FDR of 25%.

| | Random Forests | | t-test | |
|---|---|---|---|---|
| Metabolite | MDA | rank | p | FDR |
| Carnitine | 0.0090063 | 1 | 7.05E−05 | 0.020447 |
| Creatine | 0.0074746 | 2 | 0.00026849 | 0.025954 |
| 1-myristoylglycerophosphocholine | 0.0065717 | 3 | 0.014367 | 0.20832 |
| 2-stearoylglycerophosphocholine* | 0.0050097 | 4 | 0.017829 | 0.22968 |
| 2-palmitoylglycerophosphocholine* | 0.0048694 | 5 | 0.00023557 | 0.025954 |
| 1-linoleoylglycerophosphocholine | 0.0043577 | 6 | 0.0017183 | 0.062288 |
| 1-eicosatrienoylglycerophosphocholine* | 0.0037265 | 7 | 0.019709 | 0.22968 |
| Pro-hydroxy-pro | 0.0034899 | 8 | 0.024475 | 0.23013 |
| Acetylcarnitine | 0.0033807 | 9 | 0.022094 | 0.22968 |
| Indolepropionate | 0.0028582 | 10 | 0.0089222 | 0.14375 |
| Propionylcarnitine | 0.0024763 | 11 | 0.0046577 | 0.095908 |
| 1-palmitoylglycerophosphocholine | 0.0024133 | 12 | 0.0022978 | 0.074041 |
| HWESASXX* | 0.0023734 | 13 | 0.00081488 | 0.059079 |
| 7-alpha-hydroxy-3-oxo-4-cholestenoate (7-Hoca) | 0.0021501 | 14 | | |
| 2-linoleoylglycerophosphocholine* | 0.0020216 | 15 | 0.021184 | 0.22968 |
| 1-oleoylglycerophosphocholine | 0.0019344 | 16 | 0.0016981 | 0.062288 |
| Glutamate | 0.001824 | 17 | 0.0041905 | 0.095908 |
| Uridine | 0.0017756 | 18 | 0.0036407 | 0.095908 |
| 1-docosahexaenoylglycerophosphocholine* | 0.0017375 | 19 | 0.0047327 | 0.095908 |
| 1-palmitoleoylglycerophosphocholine* | 0.0016942 | 20 | 0.0010363 | 0.060106 |
| 1-eicosadienoylglycerophosphocholine* | 0.0016473 | 21 | | |
| 4-androsten-3beta,17beta-diol disulfate 1* | 0.0016376 | 22 | 0.049978 | 0.32208 |
| 4-androsten-3beta,17beta-diol disulfate 2* | 0.0015266 | 23 | 0.019038 | 0.22968 |
| 3-methylhistidine | 0.0014011 | 24 | 0.0060555 | 0.10976 |
| Octanoylcarnitine | 0.0013331 | 25 | 0.02698 | 0.23013 |
| Indoleacetate | 0.0012452 | 26 | | |
| 1-arachidonoylglycerophosphocholine* | 0.0011835 | 27 | 0.0044521 | 0.095908 |
| Pregn steroid monosulfate* | 0.0011832 | 28 | 0.029489 | 0.23884 |
| 13-HODE + 9-HODE | 0.0011674 | 29 | | |
| Butyrylcarnitine | 0.0011623 | 30 | 0.031007 | 0.23884 |
| Ornithine | 0.0010938 | 31 | | |
| Pseudouridine | 0.0010069 | 32 | 0.026032 | 0.23013 |
| Beta-hydroxyisovalerate | 0.00082584 | 33 | 0.0081443 | 0.13893 |
| Lysine | 0.00080311 | 34 | | |
| Hexanoylcarnitine | 0.00076025 | 35 | 0.029926 | 0.23884 |
| 21-hydroxypregnenolone disulfate | 0.00075953 | 36 | 0.038041 | 0.2758 |
| 1-pentadecanoylglycerophosphocholine* | 0.00075436 | 37 | | |
| Aspartate | 0.00063795 | 38 | | |
| Inosine | 0.00063086 | 39 | | |
| Catechol sulfate | 0.00059252 | 40 | 0.035063 | 0.26072 |
| Tauroursodeoxycholate | 0.00058711 | 41 | | |
| Benzoate | 0.00054853 | 42 | | |
| Glucose | 0.00053067 | 43 | | |
| Deoxycarnitine | 0.00051061 | 44 | 0.0049607 | 0.095908 |
| Tryptophan betaine | 0.00050939 | 45 | 0.023538 | 0.23013 |
| N1-methyladenosine | 0.00049879 | 46 | | |
| Caproate (6:0) | 0.00049551 | 47 | 0.046358 | 0.31264 |
| N-acetylthreonine | 0.00042984 | 48 | | |
| Testosterone sulfate | 0.00041504 | 49 | | |
| 1-stearoylglycerophosphoethanolamine | 0.00041486 | 50 | | |
| Glutaroyl carnitine | 0.00038959 | 51 | 0.02667 | 0.23013 |
| Andro steroid monosulfate 2* | 0.00034904 | 52 | 0.020785 | 0.22968 |
| Alpha-ketobutyrate | 0.00034405 | 53 | 0.031296 | 0.23884 |
| 3-hydroxykynurenine | 0.00033738 | 54 | | |
| Cysteine | 0.00032585 | 55 | | |
| 1-heptadecanoylglycerophosphocholine | 0.0003238 | 56 | | |
| Pyruvate | 0.00030816 | 57 | | |
| Urate | 0.00029893 | 58 | 0.011309 | 0.17261 |
| 1-oleoylglycerol (1-monoolein) | 0.00028817 | 59 | | |
| Octadecanedioate | 0.00027673 | 60 | | |
| Laurylcarnitine | 0.0002715 | 61 | | |
| 1-stearoylglycerophosphocholine | 0.00026693 | 62 | 0.026739 | 0.23013 |
| 2-oleoylglycerophosphocholine* | 0.00026011 | 63 | | |
| Citrulline | 0.00025617 | 64 | | |
| Mannose | 0.00023963 | 65 | | |
| Cystine | 0.00022018 | 66 | | |
| Oleoylcarnitine | 0.00020014 | 67 | 0.04174 | 0.28821 |
| Adenosine 5′-monophosphate (AMP) | 0.0001953 | 68 | 0.0013171 | 0.062288 |
| Phosphate | 0.00018954 | 69 | | |
| Margarate (17:0) | 0.00018867 | 70 | | |

### Metabolic Profile as a Biomarker of Myeloma

Having found a direct correlation in the BM PC content and metabolic score (FIG. 3D), the authors asked whether the metabolic profile could report on disease load. The commonest indirect correlate of tumor size, the M-component, was found to correlate with both BM and peripheral metabolic scores (FIG. 9A-B), as well as, expectedly[2], with BM PC counts (FIG. 9C). The authors found stronger correlations of the metabolic scores with BM PC counts than with the M-component, both for BM (rs 0.62 vs. 0.67) and peripheral (rs 0.74 vs. 0.45) profiles (FIGS. 3D and 9A-B,D), suggesting that metabolic markers may inform on tumor burden. In all, these results evidence that metabolic profiling of both BM and peripheral plasma provides information that holds potential to increase the accuracy of disease evaluation, independently and ideally in combination with the M-component.

Independent Analyses of Different Disease Vs. Control Groups Consistently Identify a Set of Myeloma-Associated Metabolic Alterations

While successfully separating disease and control samples, feature transformation-based methods are not recommended for biomarker identification[13]. In search for individual metabolites as biomarkers of MM, the authors interrogated the whole dataset performing independent comparisons by t-test (followed by multiple testing correction) between disease and control groups. The authors thus analyzed BM samples comparing all active myelomas (PRO and NEW) to all controls (MGUS and REM), and peripheral blood samples comparing SMM to HV, MGUS to NEW, and PRO to REM (FIG. 4). In this setting, no sample was shared by different analyses, and a total of 125 patients and 157 samples contributed to model the differences. As shown in the Venn diagram (FIG. 4A-B), 55/284 metabolites were found to be significantly different (p<0.05) in at least two of the disease vs. non-disease comparisons, of which 18/284 in at least three, and 4 in all four tests (Table 5).

TABLE 5
Convergent results of different MM vs. non-MM comparisons.
Comparison between the results on t-tests for unpaired variables on four non-overlapping sets of
disease vs. control groups, including BM NEW + PRO vs. MGUS + REM, peripheral blood SMM
vs. HV, REM vs. PRO peripheral blood, and MGUS vs. NEW peripheral blood samples. Results
shown as t-test p value (P) and associated FDR; grey shading highlights the four metabolites
identified by all four models (1-linoleoylglycerophosphocholine, 2-
linoleoylglycerophosphocholine, C3f, palmitoyl sphingomyelin).

| | Bone Marrow | | | | | | | |
|---|---|---|---|---|---|---|---|---|
| | NEW + PRO vs | | Peripheral blood | | | | | |
| | MGUS + REM | | SMM vs HV | | REM vs PRO | | MGUS vs NEW | |
| Metabolite | P | FDR | P | FDR | p | FDR | P | FDR |
| 1-arachidonoyl- | 5.00E−05 | 0.002 | | | 1.99E−03 | 0.061 | 1.19E−05 | 0.000 |
| glycerophosphocholine | | | | | | | | |
| 1-docosahexaenoylglycerophosphocholine | 1.37E−04 | 0.004 | | | 5.81E−03 | 0.092 | 4.76E−04 | 0.006 |
| 1-docosapentaenoyl- | 1.52E−02 | 0.134 | | | | | 1.28E−02 | 0.053 |
| glycerophosphocholine | | | | | | | | |
| 1-eicosadienoylglycerophosphocholine | 2.40E−04 | 0.006 | | | 2.40E−03 | 0.061 | 5.04E−05 | 0.001 |
| 1-eicosatrienoyl- | 1.64E−05 | 0.001 | | | 2.25E−03 | 0.061 | 3.44E−06 | 0.000 |
| glycerophosphocholine | | | | | | | | |
| 1-heptadecanoyl- | 8.89E−03 | 0.094 | | | 9.50E−04 | 0.054 | | |
| glycerophosphocholine | | | | | | | | |
| 1-linoleoylglycerophosphocholine | 1.06E−04 | 0.003 | 4.04E−02 | 0.309 | 9.21E−04 | 0.054 | 1.41E−06 | 0.000 |
| 1-linoleoyl- | 3.77E−02 | 0.216 | | | | | 1.74E−03 | 0.015 |
| glycerophosphoethanolamine | | | | | | | | |
| 1-myristoylglycerophosphocholine | 1.67E−06 | 0.000 | | | 2.60E−03 | 0.061 | 3.72E−06 | 0.000 |
| 1-oleoylglycerophosphocholine | 2.57E−05 | 0.001 | | | 8.06E−04 | 0.054 | | |
| 1-palmitoleoyl- | 4.23E−06 | 0.000 | | | 1.40E−03 | 0.057 | 1.74E−05 | 0.001 |
| glycerophosphocholine | | | | | | | | |
| 1-palmitoylglycerophosphocholine | 5.12E−05 | 0.002 | | | 8.05E−04 | 0.054 | 7.80E−07 | 0.000 |
| 1-pentadecanoyl- | 1.65E−04 | 0.004 | | | 2.04E−02 | 0.199 | | |
| glycerophosphocholine | | | | | | | | |
| 1-stearoylglycerophosphocholine | 3.14E−02 | 0.190 | | | 1.04E−02 | 0.129 | 4.32E−03 | 0.027 |
| 13-HODE + 9-HODE | 8.11E−03 | 0.092 | | | | | 1.86E−03 | 0.015 |
| 2-docosahexaenoyl- | 7.05E−03 | 0.088 | | | | | 2.92E−02 | 0.096 |
| glycerophosphoethanolamine | | | | | | | | |
| 2-ethylhexanoate | 4.90E−02 | 0.244 | | | | | 2.08E−02 | 0.074 |
| 2-linoleoylglycerophosphocholine | 1.86E−03 | 0.038 | 2.97E−03 | 0.123 | 2.32E−02 | 0.203 | 6.15E−03 | 0.034 |
| 2-palmitoylglycerophosphocholine | 2.76E−06 | 0.000 | | | 3.21E−06 | 0.001 | | |
| 2-stearoylglycerophosphocholine | 1.30E−06 | 0.000 | | | 2.00E−03 | 0.061 | 2.39E−03 | 0.019 |
| 2,3-dihydroxyisovalerate | | | 3.71E−02 | 0.302 | | | 1.11E−02 | 0.049 |
| 3-hydroxykynurenine | | | | | 4.07E−02 | 0.257 | 5.00E−02 | 0.144 |
| 3-methylhistidine | | | | | 2.24E−02 | 0.203 | 3.12E−02 | 0.101 |
| 3-phenylpropionate | 4.41E−02 | 0.236 | 6.24E−03 | 0.165 | | | 6.86E−04 | 0.008 |
| (hydrocinnamate) | | | | | | | | |
| 4-hydroxyphenylpyruvate | | | | | 1.29E−03 | 0.057 | 3.75E−02 | 0.116 |
| 7-alpha-hydroxy-3-oxo-4- | 5.95E−03 | 0.084 | | | | | 2.56E−03 | 0.019 |
| cholestenoate (7-Hoca) | | | | | | | | |
| acetylcarnitine | 1.30E−02 | 0.119 | | | | | 1.59E−07 | 0.000 |
| arachidonate (20:4n6) | | | | | 5.24E−03 | 0.092 | 9.49E−04 | 0.011 |
| C-glycosyltryptophan | | | | | 3.91E−02 | 0.252 | 1.47E−02 | 0.057 |
| carnitine | 5.61E−03 | 0.084 | | | | | 1.04E−07 | 0.000 |
| catechol sulfate | 5.35E−03 | 0.084 | | | | | 1.16E−03 | 0.012 |
| citrate | 1.30E−02 | 0.119 | | | | | 3.91E−03 | 0.025 |
| creatine | 2.73E−02 | 0.176 | | | | | 1.21E−05 | 0.000 |
| glycine | | | 2.00E−02 | 0.231 | | | 1.32E−02 | 0.054 |
| glycolithocholate sulfate | 1.77E−02 | 0.148 | | | | | 1.29E−04 | 0.003 |
| HWESASXX (C3f) | 4.86E−02 | 0.244 | 4.41E−06 | 0.001 | 3.13E−02 | 0.223 | 2.43E−05 | 0.001 |
| indolepropionate | 5.94E−03 | 0.084 | | | | | 4.25E−04 | 0.006 |
| isoleucine | | | 8.01E−03 | 0.179 | | | 2.06E−02 | 0.074 |
| N-(2-furoyl)glycine | | | 8.50E−05 | 0.007 | | | 8.87E−03 | 0.043 |
| N6-carbamoylthreonyladenosine | | | 4.23E−02 | 0.315 | 9.85E−03 | 0.129 | | |
| oleoylcarnitine | 1.02E−02 | 0.101 | | | 3.26E−02 | 0.226 | 1.45E−02 | 0.057 |
| palmitoyl sphingomyelin | 2.35E−02 | 0.167 | 2.42E−02 | 0.246 | 3.13E−02 | 0.223 | 1.01E−02 | 0.047 |
| phenyllactate (PLA) | | | 1.64E−04 | 0.010 | | | 5.63E−04 | 0.007 |
| proline | 1.03E−02 | 0.101 | | | | | 2.78E−02 | 0.094 |
| propionylcarnitine | | | 2.95E−02 | 0.266 | | | 2.80E−02 | 0.094 |
| pseudouridine | | | | | 4.79E−03 | 0.091 | 3.22E−04 | 0.005 |
| stearate (18:0) | 3.98E−02 | 0.217 | | | | | 3.21E−02 | 0.102 |
| stearidonate (18:4n3) | | | | | 3.45E−02 | 0.233 | 4.57E−02 | 0.135 |
| taurolithocholate 3-sulfate | 4.67E−02 | 0.244 | | | | | 7.78E−03 | 0.040 |
| tryptophan | 3.60E−03 | 0.068 | | | 1.45E−02 | 0.163 | 5.11E−03 | 0.030 |
| urate | | | 3.19E−02 | 0.272 | 1.61E−02 | 0.170 | 3.47E−03 | 0.023 |
| xanthosine | | | 9.29E−03 | 0.192 | | | 7.23E−03 | 0.038 |
| xylose | | | 4.44E−03 | 0.161 | | | 1.38E−02 | 0.056 |

The peptide HWESASLL invariably emerged as significantly increased in MM patients (FIG. 4A-D). Interestingly, 16 LPC (out of 17 named) were found to be significantly decreased in at least 2 comparisons, with 13 emerging in at least 3, and 2 (1-linoleoylglycerophosphocholine and 2-linoleoylglycerophosphocholine) in all comparisons. Palmitoylsphingomyelin, a phosphocholine-derived lipid raft constituent, was also significantly decreased in myeloma, as compared to control samples, in all 4 analyses (FIG. 4A-B, and Table 5).

### Detectable Levels of the C3f Peptide HWESASLL Hallmark Active Myeloma

The HWESASLL sequence identified the C3f peptide, a fragment of the C3 complement factor, CPAMD1. In the authors' series, C3f was undetectable in most healthy controls (80%) and MGUS patients (60%), but reached high levels in peripheral and BM plasma of most newly diagnosed MM (75%, FIG. 4C-D). No REM BM and only 25% of peripheral blood samples had detectable C3f, with lower levels than in NEW. SMM showed detectable C3f levels in over 75% of both peripheral and BM samples. In correlation analyses, HWESASLL emerged as the strongest single correlate of medullary PC count (rs 0.81, p=3.1E-08, FDR 4.4E-06). Increased levels of C3f, relative to very low/undetectable controls, have been reported in solid tumors, such as nasopharyngeal [21] and lung carcinoma [22], and in myeloid [23] and lymphoid [24]leukemia. Of relevance, C3f has been shown to actively modulate in vitro IGF1 signaling, microvascular endothelial cell proliferation, and enhance TGFβ-1 secretion by endothelial cells [25]. As increased BM microvascularization is relevant to MM progression[26], and TGFβ and IGF1 promote MM cell growth [8], elevated C3f may play a role in MM evolution. Thus, C3f is a candidate marker of myeloma progression.

### Reduced Levels of Lysophosphocholines Hallmark Active Myeloma Both in Peripheral and in Bone Marrow Plasma

In all, the authors' analysis identified 135 lipids, of which 2 sphingolipids and 30 lysolipids, including 17 LPC. Importantly, 16/17 LPC and 1 of 2 sphingolipids (phosphatidylcholine-related) were consistently found to be lower in myelomas than controls. FIG. 4E-F shows one exemplar LPC, 1-myristoylglycerophosphocholine, being significantly lower in both peripheral and BM plasma of MM patients relative to controls. Consistent with a tumor-site feature, greater differences emerged in BM, where the levels of 13 LPC species also inversely correlated with PC counts (rs<−0.43, p<0.03, FDR<15%). These results point to reduced LPC levels both in peripheral and BM plasma as a hallmark of MM.

### Aminoacid Metabolites Associated to Myeloma

Augmented osteoclastic activity and increased bone resorption are critical steps in myeloma development and progression[8]. In particular, bioptically increased bone resorption has been proposed to hold prognostic value for MGUS progression[27]. Hydroxyproline is a modified aminoacid of collagen, whose free levels as mono- or di-peptide are bone resorption markers [28] [29]. The authors found significantly increased levels of pro-hydroxy-proline in peripheral plasma of newly diagnosed myeloma patients as compared to all other groups (FIG. 10A). This is consistent with the reported absence of increased serum markers of bone resorption in MGUS [30], and of detectable bone disease or hypercalcemia in SMM, as well as with the adoption of anti-resorptive therapies in treated MM patients[1]. Pro-OH-proline was also higher in BM plasma relative to MGUS (FIG. 10B).

The tryptophan catabolite 3-hydroxykynurenine also emerged consistently from the authors' multivariate analyses. Following the kynurenine pathway, tryptophan is catabolized to kynurenine by indoleamine 2,3-dioxygenase (IDO1), and then converted to 3-hydroxykynurenine. Previously known only for its neurotoxic [31] and nephrotoxic [32] activity, 3-hydroxykynurenine has recently been reported to exert potent immunomodulatory functions, promoting mismatched allograft tolerance and depleting in vitro and in vivo T cells in transplanted mice[33]. Importantly, inhibitors of the kynurenin pathway have recently been shown to re-activate antitumoral immune responses[34]. The authors found increased peripheral levels of 3-hydroxykynurenine in patients with newly diagnosed, and relapsing or progressive myeloma relative to MGUS or healthy controls (FIG. 10C), providing preliminary evidence that the kynurenine pathway is activated in MM, with possible pathogenic significance. Sarcosine is an N-methyl glycine-derivative generally found at low levels in the peripheral blood of healthy individuals, recently proposed as a marker of prostate cancer[35], with cancer-promoting in vitro activities, including induction of migration, invasiveness, and up-regulation of pathogenic receptors[36] [7, 37]. The authors found sarcosine significantly higher in the peripheral blood of SMM patients relative to healthy and MGUS controls (FIG. 10D), where it was seldom detected. This data suggest a role for sarcosine early in MM development.

### Lysophosphocholines Support MM Cell Viability In Vitro

Having found reduced circulating LPC levels in MM patients, the authors asked whether LPC play a direct role on MM cells. The authors found LPC supplementation to decrease apoptosis of patient derived cells and two MM lines (FIG. 5A), and to increase viability of OPM2 cells (FIG. 5B) particularly upon serum starvation. These data reveal a previously unanticipated trophic role of LPC, whose consumption by malignant PC may help sustain lipid metabolism and membrane formation[38]. Previous reports indicate that phosphocholine administration can rescue MM cells from the mevalonate pathway inhibitor apomine[39], while a toxic alkyl-lysophospholipid analogue, edelfosine, has anti-myeloma activity[40]. In keeping with these observations, the authors' data suggest that LPC may be uptaken by MM cells, possibly entering the Lands's cycle to form phospholipids[41], and sustain membrane remodeling and biogenesis.

### Discussion

MM is characterized by diffuse and localized growth, severe systemic symptoms, resistance to conventional chemotherapy and inevitable recurrence. Standard diagnosis depends on end-organ damage, BM biopsy, and a very specific marker, the M-component, also found in MGUS [1, 2].

As most MGUS individuals will never develop MM, methods to assess potential progression need to be sustainable and efficient [2, 4].

In the present invention, the authors deployed a high throughput unbiased technique, metabolomics, to address all small metabolites in the BM and peripheral plasma of patients at different stages of MM development and progression. The metabolic profile of both peripheral and BM plasma proved able to discriminate patients with active MM from controls (FIGS. 2-3, 7-8), suggesting a strong connection with tumor load, as metabolic scores efficiently correlated with BM PC counts (FIG. 3D, 9). Different analytical methods and independent comparisons of disease vs. non disease groups converged in identifying a panel of discriminants, which often independently achieved statistical significance in univariate analysis among groups (by ANOVA and Tukey's post-hoc test, FIGS. 4, 10).

Certain metabolites, generally undetectable or found at very low levels in healthy individuals, such as sarcosine or the C3f peptide HWESASLL, were increased in the peripheral blood of patients with active, recurrent or high-risk disease (FIGS. 4, S5). These species are also increased in other tumors, and hence are not cancer type-specific [7, 21, 23, 42]. MM is characterized by an extremely PC-specific marker, the M-component, which is directly produced by the abnormal clone, but poorly predicts malignancy and time to progression[2]. The availability of novel markers, therefore, could help to monitor myeloma progression in individuals bearing precursor conditions (with detectable M-component), combining high accuracy with low costs. In light of previous reports of biological activities and of their association with MM, these molecules also merit further investigation to address their function in MM pathogenesis.

Few metabolites, like pro-hydroxy-proline and 3-hydroxykynurenine, displayed interesting intra-group heterogeneous distributions. Functional links with known pathogenic mechanisms encourage further studies in larger cohorts.

Dendritic cells (DC) from MM patients fail to induce antitumor immunity because of inhibition by TGFβ^{[43]}, which, in turn, has been shown to turn DC tolerogenic by up-regulating IDO1[44]. Moreover, mesenchymal stromal cells, known to support MM development[8], produce TGFβ, express IDOL and possess known immunomodulatory functions[45]. The authors' finding of elevated levels of 3-hydroxykynurenine in newly diagnosed and relapsed MM patients suggests a possible role of the kynurenin pathway in MM immune escape, amenable to pharmacological treatment [30].

LPC were found to be collectively (16/17) and selectively (relative to other lipids) decreased in myeloma patients (FIG. 4), and to support myeloma cell survival and growth in vitro (FIG. 5). While lipid metabolism is an emerging target in MM [35, 36], the authors' findings suggest that LPC uptake may play a role in myeloma cell biology in vivo and indicate novel potential therapeutic targets.

In all, the authors' data show that metabolomics is a feasible and powerful approach to MM, which could integrate with other technological and clinical tools to address the clinical and biological complexity of the disease.

### REFERENCES

1. Palumbo, A. and K. Anderson, The New England j. of med., 2011. 364(11): p. 1046-60.
2. Korde, N., S. Y. Kristinsson, and O. Landgren, Blood, 2011. 117(21): p. 5573-81.
3. Dimopoulos, M., et al., Blood, 2011. 117(18): p. 4701-5.
4. Landgren, O., et al., Blood, 2009. 113(22): p. 5412-7.
5. Landgren, O. and S. V. Rajkumar, Seminars in hematology, 2011. 48(1): p. 66-72.
6. Spratlin, J. L., et al., Clinical cancer research: an official journal of the American Association for Cancer Research, 2009. 15(2): p. 431-40.
7. Sreekumar, A., et al., Nature, 2009. 457(7231): p. 910-4.
8. Hideshima, T., et al., Nature reviews. Cancer, 2007. 7(8): p. 585-98.
9. Morgan, G. J., B. A. Walker, and F. E. Davies, Nature reviews. Cancer, 2012. 12(5): p. 335-48.
10. Lopez-Corral, L., et al., Clinical cancer research: an official journal of the American Association for Cancer Research, 2011. 17(7): p. 1692-700.
11. Lawton, K. A., et al., 2008. 9(4): p. 383-97.
12. Evans, A. M., et al., Analytical chemistry, 2009. 81(16): p. 6656-67.
13. Hilario, M. and A. Kalousis, Briefings in bioinformatics, 2008. 9(2): p. 102-18.
14. Rajkumar, S. V., et al., Blood, 2011. 117(18): p. 4691-5.
15. Xia, J. and D. S. Wishart, Nature protocols, 2011. 6(6): p. 743-60.
16. Blekherman, G., et al., Metabolomics: Official journal of the Metabolomic Society, 2011. 7(3): p. 329-343.
17. Xia, J., et al., Nucleic acids research, 2009. 37(Web Server issue): p. W652-60.
18. Nishiumi, S., et al., PloS one, 2012. 7(7): p. e40459.
19. Bylesjo, M., et al., Journal of Chemometrics, 2006. 20(8-10): p. 341-351.
20. Breiman, L., Machine Learning, 2001. 45(1): p. 5-32.
21. Chang, J. T., et al., Clinical biochemistry, 2006. 39(12): p. 1144-51.
22. Lin, X. L., et al., Chinese medical journal, 2010. 123(1): p. 34-9.
23. Liang, T., et al., Proteomics, 2010. 10(1): p. 90-8.
24. Ishida, Y., et al., Cancer letters, 2008. 271(1): p. 167-77.
25. Xiang, Y., et al., Arthritis and rheumatism, 2007. 56(6): p. 2018-30.
26. Vacca, A., et al., Blood, 1999. 93(9): p. 3064-73.
27. Bataille, R., D. Chappard, and M. F. Basle, Blood, 1996. 87(11): p. 4762-9.
28. Christenson, R. H., Clinical biochemistry, 1997. 30(8): p. 573-93.
29. Husek, P., et al., Clinical chemistry and laboratory medicine: CCLM FESCC, 2008. 46(10): p. 1391-7.
30. Ng, A. C., et al., Blood, 2011. 118(25): p. 6529-34.
31. Zwilling, D., et al., Cell, 2011. 145(6): p. 863-74.
32. Kikuchi, K., et al., Journal of chromatography. B, Analytical technologies in the biomedical and life sciences, 2010. 878(20): p. 1662-8.
33. Zaher, S. S., et al., Investigative ophthalmology & visual science, 2011. 52(5): p. 2640-8.
34. Liu, X., et al., Blood, 2010. 115(17): p. 3520-30.
35. Struys, E. A., et al., Annals of clinical biochemistry, 2010. 47(Pt 3): p. 282.
36. Chen, X., et al., The Journal of biological chemistry, 2011. 286(18): p. 16091-100.
37. Dahl, M., et al., Molecular biology reports, 2011. 38(7): p. 4237-43.
38. Brewer, J. W. and S. Jackowski, Biochemistry research international, 2012. 2012: p. 738471.
39. Roelofs, A. J., et al., The Journal of pharmacol. and exp. ther., 2007. 322(1): p. 228-35.
40. Mollinedo, F., et al., Oncogene, 2010. 29(26): p. 3748-57.
41. Shindou, H., et al., Journal of lipid research, 2009. 50 Suppl: p. S46-51.
42. Thysell, E., et al., PloS one, 2010. 5(12): p. e14175.
43. Brown, R. D., et al., Blood, 2001. 98(10): p. 2992-8.
44. Belladonna, M. L., et al., Journal of immunology, 2008. 181(8): p. 5194-8.
45. Opitz, C. A., et al., Stem cells, 2009. 27(4): p. 909-19.

1. A method for the prognosis of Symptomatic Multiple Myeloma (MM) and/or to monitor the response and/or the efficacy of a MM therapy comprising:
detecting and/or quantifying at least one marker selected from the group consisting of: C3f peptide or a fragment thereof, 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, Creatinine, Glutaroyl carnitine, 2-linoleoylglycerophosphocholine, N-(2-furoyl)glycine, 1-palmitoleoylglycerophosphocholine, Citrate, Carnitine, Sarcosine (N-Methylglycine), 3-hydroxykynurenine, Xanthosine, 1-docosahexaenoylglycerophosphocholine, Testosterone sulfate, 1-palmitoylglycerophosphocholine, Glycerol 3-phosphate (G3P), Acetylcarnitine, N1-methyladenosine, Pro-hydroxy-pro, Urate, 7-alpha-hydroxy-3-oxo-4-cholestenoate (7-Hoca), Cysteine, 2-hydroxybutyrate (AHB), Cortisol, 1-oleoylglycerophosphoethanolamine, Butyrylcarnitine, Pyridoxate, Pseudouridine, 1-stearoylglycerophosphocholine, Palmitoyl sphingomyelin, 1-oleoylglycerophosphocholine, Creatine, 1-docosapentaenoylglycerophosphocholine, Gamma-glutamylphenylalanine, Catechol sulfate, 13-Hydroxyoctadecadienoate (13-HODE), 9-Hydroxyoctadecadienoate (9-HODE), Hexanoylcarnitine, 2-hydroxypalmitate, Indolepropionate, Oleoylcarnitine, Succinate, Tyrosine, Levulinate (4-oxovalerate), Adenosine 5′-monophosphate (AMP), Pyroglutamine, Pregn steroid monosulfate, Alpha-hydroxyisovalerate, Andro steroid monosulfate 2,3-methylhistidine, Lactate, Caprate (10:0), 3-hydroxyisobutyrate, Scyllo-inositol, N-acetyl-beta-alanine, 2-aminobutyrate, Phenyllactate (PLA), Heptanoate (7:0), Beta-hydroxyisovalerate, 3-methoxytyrosine, Deoxycarnitine, 1-palmitoylplasmenylethanolamine, 3-methyl-2-oxobutyrate, 3-phenylpropionate (hydrocinnamate), Propionylcarnitine, Pelargonate (9:0), Tryptophan, 2-stearoylglycerophosphocholine, Pregnenolone sulfate, Phosphate, N-acetylmethionine, Caprylate (8:0), N-formylmethionine, Cyclo(leu-pro), 1-heptadecanoylglycerophosphocholine, Pregnen-diol disulfate, Acetylphosphate, Taurochenodeoxycholate, Arginine, Cholesterol, C-glycosyltryptophan, 4-androsten-3beta.17beta-diol disulfate 1, N-methyl proline, Stearoyl sphingomyelin, Mannose, 21-hydroxypregnenolone disulfate and 1-eicosadienoylglycerophosphocholine in a sample obtained from a subject;
optionally comparing the value of the quantified marker to a control value.

2. The method according to claim 1 wherein the at least one marker is the C3f peptide or a fragment thereof.

3. The method according to claim 1 wherein the at least one marker is selected from the group consisting of:
1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

4. The method according to claim 1 wherein the at least one marker is selected from the group consisting of:
the C3f peptide or a fragment thereof, 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

5. The method according to claim 1 wherein the at least one marker is selected from the group consisting of:
C3f peptide or a fragment thereof, 1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, Creatinine, Glutaroyl carnitine, 2-linoleoylglycerophosphocholine, N-(2-furoyl)glycine, 1-palmitoleoylglycerophosphocholine, Citrate, Carnitine, Sarcosine (N-Methylglycine), 3-hydroxykynurenine, Xanthosine, 1-docosahexaenoylglycerophosphocholine, Testosterone sulfate, 1-palmitoylglycerophosphocholine, Glycerol 3-phosphate (G3P), Acetylcarnitine, N1-methyladenosine, Pro-hydroxy-pro, Urate and 7-alpha-hydroxy-3-oxo-4-cholestenoate (7-Hoca).

6. The method according to claim 5 wherein the at least one marker is selected from the group consisting of:
C3f peptide or a fragment thereof, Sarcosine (N-Methylglycine), 3-hydroxykynurenine, Pro-hydroxy-pro, 1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine.

7. The method according to claim 5 wherein the at least one marker is selected from the group consisting of:
C3f peptide or a fragment thereof, Sarcosine (N-Methylglycine), 3-hydroxykynurenine and Pro-hydroxy-pro.

8. The method according to claim 1 wherein the following markers are detected and/or quantified:
the C3f peptide or a fragment thereof, 1-arachidonoylglycerophosphocholine, 1-myristoylglycerophosphocholine, 2-palmitoylglycerophosphocholine, 1-pentadecanoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine, 1-stearoylglycerophosphocholine, 1-oleoylglycerophosphocholine, 1-docosapentaenoylglycerophosphocholine, 2-stearoylglycerophosphocholine, 1-heptadecanoylglycerophosphocholine and 1-eicosadienoylglycerophosphocholine.

9. The method according to claim 1 wherein the following markers are detected and/or quantified:
C3f peptide or a fragment thereof, Sarcosine (N-Methylglycine), 3-hydroxykynurenine and Pro-hydroxy-pro.

10. The method according 9 wherein the following further markers are detected and/or quantified:
1-arachidonoylglycerophosphocholine, 1-myristoylglycerophosphocholine, 2-palmitoylglycerophosphocholine, 1-linoleoylglycerophosphocholine, 1-eicosatrienoylglycerophosphocholine, 2-linoleoylglycerophosphocholine, 1-palmitoleoylglycerophosphocholine, 1-docosahexaenoylglycerophosphocholine, 1-palmitoylglycerophosphocholine.

11. The method according to claim 1 wherein the C3f peptide fragment comprises the sequence HWESAS (aa 9 to aa 14 of SEQ ID No. 1).

12. The method according to claim 11 wherein the C3f peptide fragment consists of sequence HWESASLL (aa 9 to aa 16 of SEQ ID No. 1).

13. The method according to claim 1 wherein the sample is blood, blood plasma or bone marrow plasma.

14. The method according to claim 1 wherein the subject is affected by Monoclonal Gammopathy of Undetermined Significance (MGUS) or Asymptomatic Multiple Myeloma or Smoldering Multiple Myeloma (SMM) or Indolent Multiple Myeloma (IMM).

15. A kit for performing the method according to claim 1 comprising:
amplification and/or detecting and/or quantifying means for at least one marker as defined in claim 1; and
appropriate reagents.

16. The kit according to claim 15 for use in a method for the prognosis of Symptomatic Multiple Myeloma (MM) and/or to monitor the response and/or the efficacy of a MM therapy.

17. A microarray comprising:
solid supporting means; and
means able to detect and/or quantify at least one marker as defined in claim 1.

18. The microarray according to claim 17 for use in a method for the prognosis of Symptomatic Multiple Myeloma (MM) and/or to monitor the response and/or the efficacy of a MM therapy.
